(19) United States
(12) Patent Application Publication
(10) Pub. No.: US 2026/0286405 A1
(43) Pub. Date: Sep. 24, 2026

(54) USE OF CYCLIC AMINES IN THE PRETREATMENT OF BIOMASS

(71) Applicants: THE REGENTS OF THE UNIVERSITY OF CALIFORNIA, Oakland, CA (US); NATIONAL TECHNOLOGY & ENGINEERING SOLUTIONS OF SANDIA, LLC, Albuquerque, NM (US)

(72) Inventors: Nikhil KUMAR, Albuquerque, NM (US); Brian R. TAYLOR, Albuquerque, NM (US); Vallari CHOURASIA, Albuquerque, NM (US); Alberto RODRIGUEZ, Concord, CA (US); John M. GLADDEN, Alameda, CA (US); Blake A. SIMMONS, San Francisco, CA (US); Hemant CHOUDHARY, Emeryville, CA (US); Kenneth L. SALE, Livermore, CA (US)

(21) Appl. No.: 19/577,276

(22) Filed: Mar. 24, 2026

Related U.S. Application Data
(60) Provisional application No. 63/776,872, filed on Mar. 24, 2025.

(51) Int. Cl. C12P 13/00

(52) U.S. Cl. CPC C12P 13/001; C12P 2201/00

(57) ABSTRACT

The present invention provides for a method to deconstruct a biomass: the method comprising: (a) introducing a solvent comprising a cyclic amine, or a mixture of cyclic amines, to a biomass to dissolve at least part of solid biomass, or a part of a lignin, cellulose, and/or hemicellulose of the solid biomass, in the solvent; (b) optionally introducing an enzyme and/or a microbe to the solubilized biomass mixture such that the enzyme and/or microbe produces a sugar from the solubilized biomass mixture; (c) optionally separating the sugar from the solubilized biomass mixture; and (d) optionally separating the lignin from the solubilized biomass mixture.

### CROSS-REFERENCE TO RELATED APPLICATIONS

This application claims priority to U.S. Provisional patent application Ser. No. 63/776,872, filed Mar. 24, 2025, which is hereby incorporated by reference.

### STATEMENT OF GOVERNMENTAL SUPPORT

The invention was made with government support under Contract No. DE-AC02-05CH11231 awarded by the U.S. Department of Energy. The government has certain rights in the invention.

### THE NAMES OF THE PARTIES TO A JOINT RESEARCH AGREEMENT

The present claimed invention was generated through a joint research agreement which The Regents of the University of California and Sandia National Laboratories are party to.

### FIELD OF THE INVENTION

The present invention is in the field of biomass pretreatment.

### BACKGROUND OF THE INVENTION

Slowing the effects of climate change due to fossil fuel use requires developing alternative fuel sources that are economical and renewable. The most prevalent source of renewable carbon is lignocellulosic biomass. The holistic utilization and conversion of lignocellulosic biomass into valuable chemicals and materials is essential for the advancement of sustainable and economically viable biorefineries.¹⁻⁴ A crucial part of this process is the efficient deconstruction of the biomass, which consists of cellulose, hemicellulose, and lignin tightly bound together. A multitude of chemical, physical, and biological pretreatment methods have been devised.⁵ Most prominent pretreatment methods are chemical-based, with a variety of solvents such as alkali, dilute acids, organic solvents, ionic liquids (ILs), and deep eutectic solvents (DESs) being explored for biomass fractionation, delignification, and carbohydrate processing.⁶⁻⁸

The incorporation of pretreatment unit operations into lignocellulose conversion processes has demonstrated significant advantages in biomass fractionation and considerable benefits for subsequent processes.⁹ An optimal pretreatment solvent should fractionate diverse feedstock phenotypes (i.e., be feedstock agnostic), solubilize cellulose, hemicellulose, and lignin, and be recyclable, economical, and non-toxic to enzymes and microbial hosts.¹⁰ However, most of the documented pretreatment solvents exhibit certain deficiencies, including the generation of inhibitory byproducts and the requirement for specific operational conditions. Amine-based pretreatment solvents have garnered significant attention in lignocellulose valorization due to their a) high basicity and nucleophilicity that allows to solubilize and fractionate lignin, b) miscibility/compatibility with water, and c) ease of recovery based on boiling point.¹¹⁻¹⁴ The ester linkages between hemicellulose and lignin are selectively cleaved by amines through nucleophilic addition, leading to the incorporation of nitrogen functional groups into the structure, thereby producing lignin with unique properties. Amines possess the potential to extract lignin with favorable characteristics and facilitate the enzymatic hydrolysis of fractionated carbohydrates. Amines-based pretreatment, in conjunction with polymorphic transformation of cellulose, can markedly decrease the crystallinity index, thereby enhancing cellulose digestibility.^{12, 15}

Despite these advances, biomass pretreatment and lignocellulose fractionation remain a significant challenge as the commonly used solvents typically have limited ability to solubilize all three biomass components, high cost, low recyclability, negative environmental impacts, and a propensity to degrade useful biomolecules, rendering them unavailable for conversion to valuable fuels and chemicals.¹⁶ This limitation highlights the need for innovative solvent selection strategies for maximizing the efficacy of the solvent while minimizing environmental impacts and toxicity to enzymes and microbial hosts. Given the extremely large number of potential solvents (e.g., ILs, DES, amines, organic solvents), experimental screening is limited. Computational methods offer a promising solution to the challenge of identifying promising solvents from this huge pool of candidates. Multi-scale computational methods allow for an extensive screening of the unexplored solvent space, aiding in the discovery of efficient, non-obvious solvents based on their physicochemical properties, biomass' component (or biopolymer) solubilities, and environmental, health, and safety (EHS) attributes.¹⁷⁻²⁴

### SUMMARY OF THE INVENTION

The present invention provides for a method to deconstruct a biomass: the method comprising: (a) introducing a solvent comprising a cyclic amine, or a mixture of cyclic amines, to a biomass to dissolve at least part of solid biomass, or a part of a lignin, cellulose, and/or hemicellulose of the solid biomass, in the solvent; (b) optionally introducing an enzyme and/or a microbe to the solubilized biomass mixture such that the enzyme and/or microbe produces a sugar from the solubilized biomass mixture; (c) optionally separating the sugar from the solubilized biomass mixture; and (d) optionally separating the lignin from the solubilized biomass mixture.

In some embodiments, the solvent efficiently dissolves selective fractions of biopolymers, such as either a specific biomass component or all components simultaneously. In some embodiments, the biopolymer is lignin, cellulose and hemicellulose.

In some embodiments, the cyclic amine has a chemical structure of one of the following:

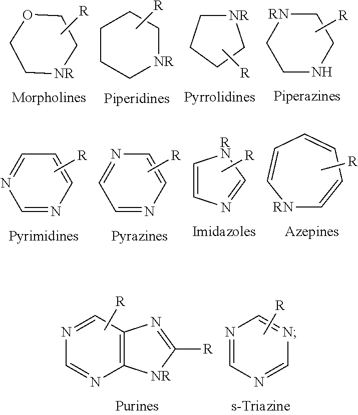

wherein R is any substituent, and each R is independent of any other R in the cyclic amine, and any 2 hydrogen atoms in a ring structure or in any R is replaced with a ═O. In some embodiments, each R is independently —H, —NH₂, alkyl, alkenyl, alkynyl, aryl, alkyl amine, alkenyl amine, alkynyl amine, or aryl amine, and optionally a ring or R comprises a carbonyl group, and optionally R is, or comprises, an amino group, carbonyl group, hydroxyl group, and/or carboxylic acid group, and optionally an oxygen atom bound to a nitrogen in the form of an oxide. When the cyclic amine is a purine, the R bonded to a nitrogen heteroatom within a ring can be bonded to any one of the four nitrogen heteroatoms.

In some embodiments, the cyclic amine comprises: (a) a 5- to 10-membered ring structure; (b) 1, 2, 3, or 4 nitrogen, oxygen, or sulfur atoms in a ring structure, wherein there is at least one nitrogen atom in the ring structure; (c) the ring structure has 0, 1, 2, 3, or 4 double-bonds; (d) none, or one or more carbon or nitrogen atoms in the ring structure is bonded to a R group, wherein each R is independently —H, —NH₂, alkyl, alkenyl, alkynyl, aryl, alkyl amine, alkenyl amine, alkynyl amine, or aryl amine, and optionally a ring or R comprises a carbonyl group, and optionally R is, or comprises, an amino group, carbonyl group, hydroxyl group, and/or carboxylic acid group; (e) optionally an oxygen atom bound to a nitrogen in the form of an oxide, and (f) optionally any carbon atom in the ring structure is bonded to a ═O.

In some embodiments, the cyclic amine has a chemical structure of:

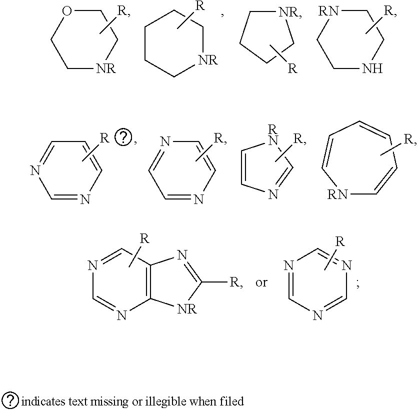

wherein each R is independent of any other R in the cyclic amine, and any 2 hydrogen atoms in a ring structure or in any R is replaced with a ═O. In some embodiments, each R is independently —H, —NH₂, alkyl, alkenyl, alkynyl, aryl, alkyl amine, alkenyl amine, alkynyl amine, or aryl amine, and optionally a ring or R comprises a carbonyl group, and optionally R is, or comprises, an amino group, carbonyl group, hydroxyl group, and/or carboxylic acid group, and when the cyclic amine is a purine, the R bonded to a nitrogen heteroatom within a ring can be bonded to any one of four nitrogen heteroatoms.

In some embodiments, each alkyl, alkenyl, alkynyl, aryl, alkyl amine, alkenyl amine, alkynyl amine, or aryl amine independently comprises at least 1, 2, 3, 4, 5, 6, 7, 8, 9, or 10 carbon atoms total, or has a longest chain having at least 1, 2, 3, 4, 5, 6, 7, 8, 9, or 10 carbon or nitrogen atoms.

In some embodiments, the cyclic amine comprises a 5, 6, 7, 8, 9, or 10-membered ring structure. In some embodiments, the cyclic amine comprises 1, 2, 3, or 4 non-carbon atoms in the ring structure. In some embodiments, the non-carbon atoms comprises a nitrogen, oxygen, or sulfur atom. In some embodiments, the cyclic amine comprises one nitrogen atom, two nitrogen atoms, or one nitrogen and one oxygen atom in the ring structure. In some embodiments, the rings comprises 1, 2, 3, or 4 double-bonds in the ring structure. In some embodiments, the cyclic amine is a Brønsted or Lewis base, and/or the cyclic amine is a hydrogen bond donor and/or acceptor. In some embodiments, the cyclic amine is any cyclic amine described herein, such as in FIGS. 5, 7, and 8.

The present invention comprises the use of specific solvents, comprising a cyclic amine, that can efficiently dissolve selective fractions of biopolymers either a specific biomass component or all components simultaneously such as lignin, cellulose and hemicellulose. The present invention demonstrates an effective framework for discovering and predicting high-performing solvents for the deconstruction of either a specific biomass components or different pair of lignocellulosic biomass fractions simultaneously. A quantum mechanics-based solvent screening framework can be used to identify solvents and cyclic amines for the improved processing of lignocellulosic biomass. In some embodiments, the cyclic amine comprises a secondary or tertiary amine with one or more nitrogen atoms. Cyclic amines, secondary or tertiary amines with one or more nitrogen atoms, are attractive due to their high nucleophilicity and basicity, higher stability, availability (lower cost) and tunability compared to primary amines.

Example 1 herein provides a thorough computational screening of over 650 unexplored cyclic amine solvent candidates to identify specific solvents that can help to dissolve either a specific biomass component or particular set of biopolymers components/all components simultaneously. Solvents are listed for each purpose mainly for comprehensive dissolution of biomass or best solvents for each category of either lignin-cellulose or lignin-hemicellulose pair. The list also includes the solvents good for selective lignin extraction (i.e. high lignin dissolution or low cellulose). The computational approach employed rigorously evaluated representative biomolecules to predict the activity coefficient of biomass fractions using the COSMO-RS model, which can later be verified experimentally. For example, 1-piperazineethanamine (PzEtN) was discovered to be one of the best solvents for selectively extracting lignin. Similarly, 1-methylpiperidine-1-oxide interacts most strongly with lignin and cellulose fractions, while 1-methylimidazole interacts most strongly with comprehensive dissolution of niomass.

The identification of unexplored cyclic amines that exhibited elevated lignin extraction (97.1%) was exceptionally higher when compared to other reported solvents. The PzEtN higher lignin removal may be more advantageous in lignin-first biorefinery approaches requiring almost complete delignification. As a future perspective, the feasibility and sustainability of the identified solvents in biorefinery processes, including fractionation and downstream processing, should be assessed.

In some embodiments, the solvent further comprises one or more polyamine as taught in U.S. Pat. No. 11,804,620, and U.S. Patent Application Publication No. 2024/0199512, both of which are incorporated by reference.

In some embodiments, the solvent has a viscosity having a value equal to or less than about 0.001 cP, 0.01 cp, 0.1 cP, 1 cP, 10 cP, 20 cP, 30 cP, 40 cP, or 50 cP, or within a range of any two of the preceding values, at a temperature of about 25° C. In some embodiments, the solvent has a viscosity having a value equal to or less than about 0.001 cP, 0.01 cp, 0.1 cP, 1 cP, 10, cP, 50 cP, 100 cP, 150 cP, 200 cP, 250 cP, 300 cP, 350 cP, 400 cP, 450 cP, 500 cP, 550 cP, or 600 cP, or within a range of any two of the preceding values, at a temperature of about 90° C. In some embodiments, the solvent has a viscosity having a value equal to or less than about 40 cP, 45 cp, 50 cP, 55 cP, or 60 cP at a temperature of about 90° C.

In some embodiments, the solvent has a boiling point having a value equal to or less than about 40° C., 50° C., 60° C., 70° C., 80° C., 90° C., 100° C., 110° C., 120° C., 130° C., 140° C., 150° C., 160° C., 170° C., 180° C., 190° C., 200° C., 210° C., 220° C., 230° C., 240° C., 250° C., 260° ³C, 270° C., 280° C., 290° C., 300° C., or 310° C., or within a range of any two of the preceding values.

In some embodiments, the solvent or cyclic amine has excess enthalpy having a H^{E} values equal to or more than about −1.8, −1.7, −1.6, −1.5, −1.4, −1.3, or −1.2, or within a range of any two of the preceding values. Hansen solubility parameter and thermodynamic parameters (excess enthalpy and activity coefficient) has been identified as an effective lignin solvent (with an excess enthalpy, H^{E} value ≥−1.5)

In some embodiments, the method further comprises recovering the cyclic amine from the solvent, such as through low pressure or vacuum distillation. In some embodiments, the method further comprises separating the cyclic amine from the solubilized biomass mixture by distillation. In some embodiments, the distillation is low pressure distillation or vacuum distillation.

In some embodiments, the solvent further comprises an IL, or components thereof, and/or components that can form a deep eutectic solvent (DES).

In some embodiments, the one or more individual components are selected from the group consisting of molecules that can form ILs: cations (such as an amine containing molecules such as ethanolamine, choline, and the like) and anions (such as mineral and organic acids, such as sulfuric acid, acetic acid, and the like). In some embodiments, the introducing step (a) comprises introducing two or individual components to the biomass, wherein the two or individual components form an IL, or mixture thereof. In some embodiments, the components already present in the biomass are components that are naturally found in a biomass.

In some embodiments, the one or more individual components are selected from the group consisting of molecules that can form DES, such as halide and organic salts (such as choline chloride, zinc chloride, ammonium acetate, and the like), organic acids (such as acetic, lactic, tartaric, etc.), polyols (such as ethylene glycol, propanediol, glycerol, glucose, etc.), amines (such as urea, acetamine, thiourea, and the like).

In some embodiments, the introducing step (a) comprises introducing two or individual components to the biomass, wherein the two or individual components form a DES, or mixture thereof. In some embodiments, the introducing step (a) comprises introducing each individual component separately to the biomass.

In some embodiments, the method further comprises (b) introducing an enzyme and/or a microbe to the solubilized biomass mixture such that the enzyme and/or microbe produces a sugar from the solubilized biomass mixture. In some embodiments, the method further comprises (c) separating the sugar from the solubilized biomass mixture. In some embodiments, the method results in a yield of equal to or more than about 80%, 85%, 90%, or 95% of sugar from the biomass.

In some embodiments, step (a) does not comprise, or lacks, introducing or adding any water to the biomass or mixture. In some embodiments, the amount of water in the mixture, excluding or including water or moisture naturally found in the biomass is no more than about 10%, 9%, 8%, 7%, 6%, 5%, 4%, 3%, 2%, 1%, 0.5%, or 0.1% by weight or volume of the mixture.

The present invention provides for compositions and methods described herein. In some embodiments, the compositions and methods further comprise steps, features, and/or elements described in U.S. patent application Ser. No. 16/737,724, hereby incorporated by reference in its entirety.

In some embodiments, the method, or one-pot method, does not require any solid-liquid separation step. In some embodiments, the one-pot method does not require adjustment of the pH level in the one-pot composition. In some embodiments, the one-pot method does not require any dilution, or addition of water or medium, after pretreatment and/or before saccharification and fermentation. In some embodiments, the reaction of the enzyme and the growth of the microbe occur in the same one-pot composition. In some embodiments, the cyclic amine, IL, DES, or mixture thereof, is renewable as it can be continuous in use. In some embodiments, the one-pot method can produce a yield of sugar that is equal to or more than about 50%, 60%, 70%, 75%, or 80%, or any other value described herein.

In some embodiments, using bio-compatible solvents enables a one-pot biomass conversion which eliminates the needs of mass transfer between reactors and the separation of solid and liquid. In some embodiments, the method does not require recycling any catalyst and/or enzyme. In some embodiments, the method requires less water usage than current biomass pretreatment. The method can produce fuels/chemicals at a higher titer and/or yield in a single vessel without any need for intermediate units of mass transfer and/or solid/liquid separation.

Cyclic amines that have a ln(γ) of a biopolymer (such as lignin, cellulose, or hemicellulose) that is less than zero is good as solvents for that biopolymer. In some embodiments, the cyclic amine has a ln(γ) of a biopolymer (such as lignin, cellulose, or hemicellulose) that is (or less than) about 0.0, −0.1, −0.2, −0.3, −0.4, −0.5, −0.6, −0.7, −0.8, −0.9, −1.0, −1.1, −1.2, −1.3, −1.4, −1.5, −1.6, −1.7, −1.8, −1.9, −2.0, −2.1, −2.2, −2.3, −2.4, −2.5, −2.6, −2.7, −2.8,-2.9, −3.0, −3.1, −3.2, −3.3, −3.4, −3.5, −3.6, −3.7, −3.8, −3.9, or −4.0, or a value within a range of two preceding values thereof. In some embodiments, the cyclic amine is a good solvent (i.e., a ln(γ) less than zero) for one biopolymer, but is a bad solvent (i.e., a ln(γ) greater than zero) for another biopolymer. In some embodiments, the cyclic amine is a good solvent for lignin, but is a bad solvent for cellulose. In some embodiments, the cyclic amine is a good solvent for cellulose, but is a bad solvent for lignin. In some embodiments, the cyclic amine is a good solvent for lignin and cellulose.

The following are predictive rules for Cellulose Dissolution: 1. The presence of oxygen in the cyclic amine structure is a favorable factor in the design of cyclic amines as solvents for pretreatment of lignocellulosic biomass to extract the cellulose from it. 1a. For instance, cellulose is selectively extracted from biomass if the oxygen is bound to the nitrogen in the form of oxide. For example, the following cyclic amines have a higher affinity for cellulose:

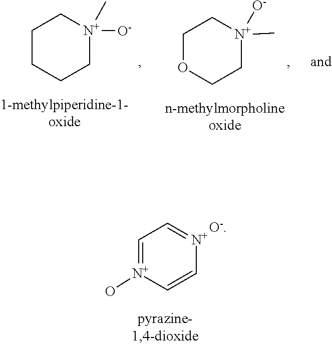

1b. Also, the presence of oxygen as carbonyl group (C═O) as a structural motif in the cyclic amine is favorable for cellulose dissolution. For example, the following five and six membered ring compounds cyclic amines have a higher affinity for cellulose:

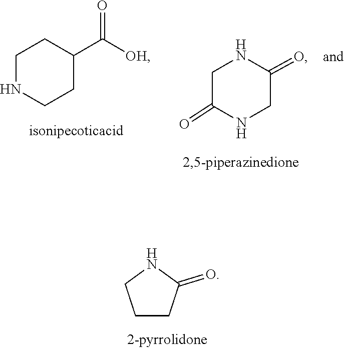 [SMILES: O=C(O)C1CCNCC1 ; O=C1CNC(=O)CN1 ; "and" ; O=C1CCCN1]

1c. Additionally, an oxygen atom present as a ring-heteroatom is unfavorable for effective cellulose dissolution. For example, the following cyclic amine has less affinity for cellulose dissolution:

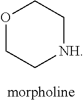

2. If there are no oxygen atoms in the ring, it has been observed that cyclic amines require at least two or more nitrogen atoms in five or six membered rings. Examples of such cyclic amines are:

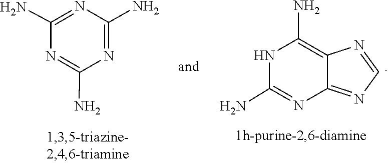 [SMILES: Nc1nc(N)nc(N)n1 ; Nc1nc2ncnc-2c(N)[nH]1]

3. The solvents that exhibit exceptional cellulose solubility are also readily soluble in water.

The following are predictive rules for Lignin Dissolution: 1. The amine solvents with pKa values in range of about 8 and about 15 are the effective for selective lignin extraction, and more precisely, solvents with pKa values between about 9 and about 12 are found to be excellent for selective lignin extraction. 2. The efficiency of cyclic amines in selective extraction of lignin from lignocellulosic biomass is significantly low if their pKa values are less than 3.

TABLE 2
List of solvents for cellulose dissolution at 140° C. and 1 atm pressure.

| S. No. | Compound Name | ln(gamma)_Cellulose | B.P. (° C.) | M.P.(° C.) | Cost/ton |
|---|---|---|---|---|---|
| 1 | 1-methylpiperidine- | −3.67765338 | 304 | N/A | $1200- |
| | 1-oxide | | | | 1500 |
| 2 | isonipecoticacid/4- | −2.67806324 | 265.8 ± 33.0 | 336 | $1000- |
| | Piperidinecarboxylic | | | | 2000 |
| | acid | | | | |
| 3 | 1,3,5-triazine-2,4,6- | −1.12375128 | NA | 343 | $1,100.00- |
| | triamine | | | | $1,550.00 |
| 4 | 2,5-piperazinedione | −0.98240439 | NA | 300 | $1,200.00- |
| | | | | | $1,500.00 |
| 5 | 2-pyrrolidone | −0.89243394 | 251 | −23 | $1,100.00- |
| | | | | | $1,500.00 |
| 6 | pyridazine | −0.48431262 | 208 | −8 | $2600- |
| | | | | | 2800 |
| 7 | piperazine | −0.47816 | 146-148 | 106 | $374- |
| | | | | | $384 |
| 8 | 1-methylimidazol | −0.38357672 | 199 | −6 | $1500- |
| | | | | | $2000 |
| 9 | n-methyl-2- | −0.3421156 | 198.7 ± 9.0 | −24 | $1500- |
| | pyrrolidinone | | (° C.) | | 200 |

In some embodiments, the present invention is useful for the pulp and paper industry to extract clean cellulose from lignocellulosic biomass. In some embodiments, the solvent can be recovered using vacuum distillation. The best solvents for clean cellulose extraction are listed in Table 2. In some embodiments, the cyclic amine is 1-piperazineethanamine (PzEtN), which is a very effective solvent for selective lignin extraction and has the highest lignin removal (about 97.1%) experimentally.

In some embodiments, the method is specifically to favor dissolving cellulose over dissolving lignin. In some embodiments, the introducing step comprises dissolving at least part of cellulose of the solid biomass, and the cyclic amine comprises (1) an oxygen atom bound to a nitrogen in the form of an oxide, (2) a carbonyl group, (3) there is no oxygen atom present as a ring-heteroatom, and/or (4) a five or six membered ring comprising two or more nitrogen atoms as ring-heteroatoms; and/or the cyclic amine is soluble in water.

In some embodiments, the cyclic amine comprises (1) an oxygen atom bound to a nitrogen in the form of an oxide, or (2) a carbonyl group. In some embodiments, the cyclic amine comprises an oxygen atom bound to a nitrogen in the form of an oxide. In some embodiments, the cyclic amine is

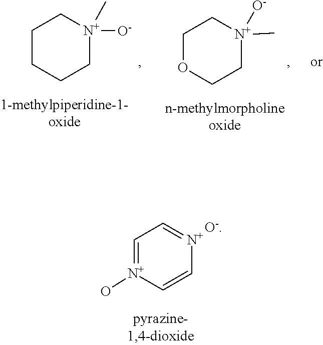

In some embodiments, the cyclic amine comprises a carbonyl group. In some embodiments, the cyclic amine is a 5-member or 6-member ring. In some embodiments, the cyclic amine is

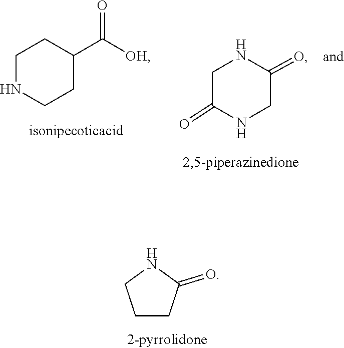 [SMILES: O=C(O)C1CCNCC1 ; O=C1CNC(=O)CN1 ; "and" ; O=C1CCCN1]

In some embodiments, the cyclic amine has no oxygen atom present as a ring-heteroatom. In some embodiments, the cyclic amine comprises a five or six membered ring comprising two or more nitrogen atoms as ring-heteroatoms. In some embodiments, the cyclic amine is

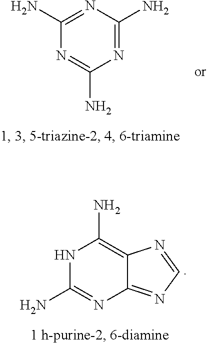 [SMILES: Nc1nc(N)nc(N)n1 ; Nc1nc2ncnc-2c(N)[nH]1]

In some embodiments, the cyclic amine is soluble in water.

In some embodiments, the method is specifically to favor dissolving lignin over dissolving cellulose. In some embodiments, the introducing step comprises dissolving at least part of lignin of the solid biomass, and the cyclic amine has a pKa value within the range of about 8 to about 15. In some embodiments, the cyclic amine has a pKa value within the range of about 9 to about 12.

The present invention provides for compositions and methods described herein.

The present invention can be used in the following: converting waste biomass (from agricultural residues, wood/paper/pulping, grasses) into biofuels, feed, and/or fiber. In some embodiments, the method achieves an improved processing of lignocellulosic biomass by treating fractions of biomass with different sets of reported solvents.

The present invention has one or more of the following advantages: (1) Comprehensive dissolution of Lignin, Cellulose and Hemicellulose fraction of lignocellulosic biomass. (2) The identification of best solvents for the solvation of Lignin-Cellulose or Lignin-Hemicellulose components pair. (3) Selective lignin extraction (high lignin solubility but very low cellulose/hemicellulose solubility). (4) Recycling and recovery via vacuum distillation. (5) Toxicity Analysis.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Classification of identified solvents in different classes of amines, where R can be hydrogen or any variety of carbon-, oxygen-, nitrogen-, or halogen-based substituent.

FIG. 1B. Workflow of the proposed methodology. (a) Data was collected from literature on amines used as solvents; (b) the COSMO-RS model was applied to a diverse dataset containing 650 cyclic amine structures by first optimizing and generating the cosmo file; (c) the model structure of different biomass fractions, mainly cellulose, hemicellulose, and lignin were generated; (d) the activity coefficients of different biomass fractions were calculated for the different cyclic amines; (e) solvents were ranked based on the solute-solvent activity coefficient; (f) solvents predicted to be the most effective were selected for further study and experimental testing.

FIG. 2. The sigma profiles of the representative biomolecules of biomass fractions (a) Lignin, (b) Cellulose, and (c) Hemicellulose calculated using the COSMO-RS model.

FIG. 3. The comprehensive interaction of lignin, cellulose, and hemicellulose fractions with solvents at 140° C. and 1 atm pressure.

FIG. 4. The combined interaction plot for cellulose and lignin with the cyclic amine at 140° C. and 1 atm pressure.

FIG. 5. The list of identified solvents selected for the selective lignin extraction at 140° C. and 1 atm pressure with the identified class of cyclic amines as depicted in FIG. 1A.

FIG. 6. COSMO-RS predicted sigma potentials for GGE, CLB, and GLX and the cyclic amines MeIM and PzEtN.

FIG. 7. The optimized structures of GGE, CLB, and GLX with cyclic amines PzEtN and MeIm along with the respective interaction energy of each complex. The dotted lines represent the H-bonds and the respective bond lengths in Angstroms(Å). C is represented by gray, O by red, N by blue, and H by white in the designated color scheme.

FIG. 8. RDG scatter and NCI plots for 1-piperazineethanamine (PzEtN) and 1-methylimidazole (MeIm) solvents with GGE, CLB, and GLX. The RDG/NCI plots are colored on a blue-green-red scale according to values of sign(2)ρ(r), ranging −0.045 and 0.025. Red indicates steric repulsion, green indicates vdW interaction, and blue indicates strong attractive interactions.

FIG. 9: X-ray diffraction patterns of sorghum biomass before and after pretreatment with respective percentage of crystallinity index.

FIG. 10: xxx. The scatter plot (FIG. 10) illustrates the thermodynamic relationship between cellulose and lignin for the top 40 candidate solvents, specifically those characterized by negative activity coefficients for cellulose (ln(γ)_{Cellulose}<0). By mapping ln(7)_{Cellulose} against ln(γ)_{Lignin}, the visualization identifies solvents capable of selective biomass fractionation. Solvents positioned in the upper-left region, such as 1-methylpiperidine-1-oxide, isonipecoticacid, and 1,3,5-triazine-2,4,6-triamine, exhibit superior performance by maximizing cellulose solubility while minimizing lignin interaction.

FIG. 11: This plot represents individual activity coefficients ln(γ), where more negative values signify higher solubility. Intersecting these bars is a green line representing the selectivity ratio

$$( \frac{{ln ( γ )}_{Cellulose}}{{ln ( γ )}_{Lignin}} )$$

offering a direct metric for fractionation efficiency. Solvents like 2,5-piperazinedione demonstrate a distinct “lignin-positive” profile, indicating they repel lignin while effectively dissolving cellulose.

FIG. 12: Lignin removal was more effective in sorghum, a representative grassy biomass, compared to pine, a softwood biomass, under pretreatment with piperazineethanamine at 40° C. Additionally, the use of an aqueous cyclic amine system resulted in slightly enhanced lignin removal and improved sugar conversion following enzymatic hydrolysis relative to neat PzEtN. Overall sugar yields, particularly glucose and xylose, remained significantly low for pine under these conditions.

FIG. 13: Sorghum exhibited substantial lignin removal, reaching approximately 67% even at the mild temperature of 40° C., demonstrating the strong potential of piperazineethanamine-based pretreatment for efficient processing of herbaceous biomass.

FIG. S1. The combined interaction for hemicellulose and lignin for cyclic amine at 140° C. and 1 atm pressure.

FIG. S2. The combined interaction for hemicellulose and cellulose for cyclic amine at 140° C. and 1 atm pressure.

FIG. S3. Appearance of sorghum biomass before and after pretreatment with two studied cyclic amines.

FIG. S4. Table S1. Boiling points and GHS hazard classifications. Boiling points were obtained from ChemSpider (averaged from multiple measurements at standard pressure), and hazard data were retrieved from PubChem. X indicates the presence of the specified hazardous classification for each compound according to GHS criteria.

FIG. S5. Table S2. COSMO surface images of all the Biopolymers used in the present study.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The present invention is a unique approach to biomass pretreatment involving the use of cyclic amines for the deconstruction of lignocellulosic biomass. Depending on the specific cyclic amine being utilized, desired physical properties such as low viscosity, low to medium boiling point can also be leveraged to enable the use of environmentally benign conditions for effective lignin removal. In some embodiments, the cyclic amine is capable of effectively pretreating biomass in order to selectively extract about 50%, 60%, 70%, or 80% lignin, while releasing about equal to or more than about 50%, 60%, 70%, 80%, or 90% sugars from the pretreated biomass. This represents an at least about 1-, 2-, 3-, 4-, or 5-fold increase in sugar release compared to the untreated biomass. This approach enables the cost-effective production of fermentable sugars and lignin—a major hurdle for producing commercially viable bioenergy from waste biomass.

This present invention provides for an approach to biomass pretreatment involving the use of cyclic amine for the deconstruction of different kinds of biomass into fermentable sugars and lignin.

Depending on the number of carbons on the backbone, and/or the isomeric conformation adopted, several key properties can be leveraged related to their performance as effective pretreatment solvents: (1) The ability to function as either Brønsted or Lewis base. (2) The ability to function as a Hydrogen bond donor and/or acceptor. (3) Low to medium Boiling Point for easy recovery via distillation. (4) Low viscosity.

In some embodiments, the cyclic amine is capable of effectively pretreating biomass (2 mm Sorghum, 140° C., 3 h, 20% solids loading) by selectively extracting lignin. In some embodiments, these chemicals can remove lignin from the raw biomass at a rate equal to or more than about 50%, 60%, 70%, or 80% while leaving about equal to or more than about 50%, 60%, 70%, 80%, or 90% of the hemicellulose and cellulose present. In some embodiments, the pretreated biomass (when hydrolyzed using 10 mg Ctec3/Htec3 9/1 per gram of biomass) can release about equal to or more than about 50%, 60%, 70%, 80%, or 90% sugars (based on a total process yield)—which can represent an at least about 1-, 2-, 3-, 4-, or 5-fold increase in sugar release compared to the untreated biomass.

This presents an opportunity for developing selective biomass fractionation techniques. The potential uses for this invention could include converting waste biomass (from agricultural residues, wood/paper/pulping, grasses) into biofuels and/or bioproducts. This process helps in achieving high concentration of fermentable sugars while leaving the residual lignin for valuable chemicals.

In some embodiments, the introducing step (a) comprises contacting a biomass and one or more individual components of an IL and/or DES. In some embodiments, the contacting step comprises introducing, adding and/or mixing the biomass with the one or more individual components of an IL and/or DES, or vice versa.

In some embodiments, the introducing one or more individual components of an IL and/or DES to a biomass takes place in a vessel and homogenized. In some embodiments, the loading is solid loading and controlled at about 5%, 10%, 15%, 20%, 25%, 30%, 35%, or 40%, or a range within any two preceding values. In some embodiments, the biomass and IL and/or DES components are heated, such as to 100° C., 110° C., 120° C., 130° C., 140° C., 150° C., 160° C., 170° C., 180° C., 190° C., 200° C., 200° C., 212° C., or a range within any two preceding values, for a period of time, such as about 1 h, 2 h, 3 h, 4 h, or 5 h, or a range within any two preceding values. In some embodiments, after pretreatment, the mixture is cooled, such as for a period of about at least 30 mins, such as at room temperature, or about 25° C., and/or then washed at least about 1×, 2×, 3×, 4×, or 5× with water, such as deionized water. In some embodiments, the resulting solid is recovered, such as separating the solid portion with the liquid portion.

In some embodiments, the biomass is a lignocellulosic biomass. In some embodiments, the vessel is made of a material that is inert, such as stainless steel or glass, that does not react or interfere with the reactions in the pretreatment mixture.

In some embodiments, the method uses a one-pot methodology, for example, using method steps and compositions as taught in U.S. patent application Ser. No. 16/737,724 (which is incorporated by reference). In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the method produces a sugar and a lignin from the biomass. The lignin can further be processed to produce an IL or DES. The sugar is used for growth by the microbe.

In some embodiments, the solubilizing is full, near full (such as at least about 70, 80, or 90%), or partial (such as at least about 10, 20, 30, 40, 50, or 60%). In some embodiments, the one-pot composition is a slurry. When the step (a), and optionally steps (c) and/or (d), are continuous, the one-pot composition is in a steady state.

### Ionic Liquid

Ionic liquids (ILs) are salts that are liquids rather than crystals at room temperatures. It will be readily apparent to those of skill that numerous ILs can be used in the present invention. In some embodiments of the invention, the IL is suitable for pretreatment of the biomass and for the hydrolysis of cellulose by thermostable cellulase. Suitable ILs are taught in ChemFiles (2006) 6(9) (which are commercially available from Sigma-Aldrich, Milwaukee, Wis.). Such suitable ILs include, but are not limited to, 1-alkyl-3-alkylimidazolium alkanoate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, wherein an “alkyl” is an alkyl group comprising from 1 to 10 carbon atoms, and an “alkanoate” is an alkanoate comprising from 1 to 10 carbon atoms. In some embodiments, the “alkyl” is an alkyl group comprising from 1 to 4 carbon atoms. In some embodiments, the “alkyl” is a methyl group, ethyl group or butyl group. In some embodiments, the “alkanoate” is an alkanoate comprising from 1 to 4 carbon atoms. In some embodiments, the “alkanoate” is an acetate. In some embodiments, the halide is chloride.

In some embodiments, the IL includes, but is not limited to, 1-ethyl-3-methylimidazolium acetate (EMIN Acetate), 1-ethyl-3-methylimidazolium chloride (EMIN Cl), 1-ethyl-3-methylimidazolium hydrogensulfate (EMIM HOSO₃), 1-ethyl-3-methylimidazolium methylsulfate (EMIM MeOSO₃), 1-ethyl-3-methylimidazolium ethylsulfate (EMIM EtOSO₃), 1-ethyl-3-methylimidazolium methanesulfonate (EMIM MeSO₃), 1-ethyl-3-methylimidazolium tetrachloroaluminate (EMIM AlCl₄), 1-ethyl-3-methylimidazolium thiocyanate (EMIM SCN), 1-butyl-3-methylimidazolium acetate (BMIM Acetate), 1-butyl-3-methylimidazolium chloride (BMIM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AlCl₄), 1-butyl-3-methylimidazolium thiocyanate (BMIM SCN), 1-ethyl-2,3-dimethylimidazolium ethylsulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, and the like.

In some embodiments, the ionic liquid is a chloride ionic liquid. In other embodiments, the ionic liquid is an imidazolium salt. In still other embodiments, the ionic liquid is a 1-alkyl-3-imidazolium chloride, such as 1-ethyl-3-methylimidazolium chloride or 1-butyl-3-methylimidazolium chloride.

In some embodiments, the ionic liquids used in the invention are pyridinium salts, pyridazinium salts, pyridium salts, pyrazinium salts, imidazolium salts, pyrazolium salts, oxazolium salts, 1,2,3-triazolium salts, 1,2,4-triazolium salts, thiazolium salts, isoquinolium salts, quinolinium salts isoquinolinium salts, piperidinium salts and pyrrolidinium salts. Exemplary anions of the ionic liquid include, but are not limited to halogens (e.g., chloride, fluoride, bromide and iodide), pseudohalogens (e.g., azide and isocyanate), alkyl carboxylate, sulfonate, acetate and alkyl phosphate.

Additional ILs suitable for use in the present invention are described in U.S. Pat. Nos. 6,177,575; 9,765,044; and, 10,155,735; U.S. Patent Application Publication Nos. 2004/0097755 and 2010/0196967; and, PCT International Patent Application Nos. PCT/US2015/058472, PCT/US2016/063694, PCT/US2017/067737, and PCT/US2017/036438 (all of which are incorporated in their entireties by reference). It will be appreciated by those of skill in the art that others ILs that will be useful in the process of the present invention are currently being developed or will be developed in the future, and the present invention contemplates their future use. The ionic liquid can comprise one or a mixture of the compounds.

In some embodiments, the IL is a protic ionic liquid (PIL). Suitable protic ionic liquids (PILs) include fused salts with a melting point less than 100° C. with salts that have higher melting points referred to as molten salts. Suitable PPILs are disclosed in Greaves et al. “Protic Ionic Liquids: Properties and Applications” Chem. Rev. 108(1):206-237 (2008). PILs can be prepared by the neutralization reaction of certain Brønsted acids and Brønsted bases (generally from primary, secondary or tertiary amines, which are alkaline) and the fundamental feature of these kinds of ILs is that their cations have at least one available proton to form hydrogen bond with anions. In some embodiments, the protic ionic liquids (PILs) are formed from the combination of organic ammonium-based cations and organic carboxylic acid-based anions. PILs are acid-base conjugate ILs that can be synthesized via the direct addition of their acid and base precursors. In some embodiments, the PIL is a hydroxyalkylammonium carboxylate. In some embodiments, the hydroxyalkylammonium comprises a straight or branched C1, C2, C3, C4, C5, C6, C7, C8, C9, or C10 chain. In some embodiments, the carboxylate comprises a straight or branched C1, C2, C3, C4, C5, C6, C7, C8, C9, or C10 chain. In some embodiments, the carboxylate is substituted with one or more hydroxyl groups. In some embodiments, the PIL is a hydroxyethylammonium acetate.

In some embodiments, the protic ionic liquid (PIL) is disclosed by U.S. Patent Application Publication No. 2004/0097755, hereby incorporated by reference.

Suitable salts for the method include combinations of organic ammonium-based cations (such as ammonium, hydroxyalkylammonium, or dimethyloleylammonium) with organic carboxylic acid-based anions (such as acetic acid derivatives (C1-C8), lactic acid, glycolic acid, and DESs such as ammonium acetate/lactic acid).

Suitable IL, such as distillable IL, are disclosed in Chen et al. “Distillable Ionic Liquids: reversible Amide O Alkylation”, Angewandte Comm. 52:13392-13396 (2013), King et al. “Distillable Acid-Base Conjugate Ionic Liquids for Cellulose Dissolution and Processing”, Angewandte Comm. 50:6301-6305 (2011), and Vijayaraghavan et al. “CO₂-based Alkyl Carbamate Ionic Liquids as Distillable Extraction Solvents”, ACS Sustainable Chem. Engin. 2:31724-1728 (2014), all of which are hereby incorporated by reference.

Suitable PIL, such as distillable PIL, are disclosed in Idris et al. “Distillable Protic Ionic Liquids for Keratin Dissolution and Recovery”, ACS Sustainable Chem. Engin. 2:1888-1894 (2014) and Sun et al. “One-pot integrated biofuel production using low-cost biocompatible protic ionic liquids”, Green Chem. 19(13):3152-3163 (2017), all of which are hereby incorporated by reference.

In some embodiments, the PILs are formed with the combination of organic ammonium-based cations and organic carboxylic acid-based anions. PILs are acid-base conjugate ILs that can be synthesized via the direct addition of their acid and base precursors. Additionally, when sufficient energy is employed, they can dissociate back into their neutral acid and base precursors, while the PILs are re-formed upon cooling. This presents a suitable way to recover and recycle the ILs after their application. In some embodiments, the PIL (such as hydroxyethylammonium acetate—[Eth][OAc]) is an effective solvent for biomass pretreatment and is also relatively cheap due to its ease of synthesis (Sun et al., Green Chem. 19(13):3152-3163 (2017)).

### Deep Eutectic Solvent (Des)

DESs are systems formed from a eutectic mixture of Lewis or Brønsted acids and bases which can contain a variety of anionic and/or cationic species. DESs can form a eutectic point in a two-component phase system. DESs are formed by complexation of quaternary ammonium salts (such as, choline chloride) with hydrogen bond donors (HBD) such as amines, amides, alcohols, or carboxylic acids. The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like. In some embodiments, the DES is any combination of Lewis or Brønsted acid and base. In some embodiments, the Lewis or Brønsted acid and base combination used is distillable.

In some embodiments, DES is prepared using an alcohol (such as glycerol or ethylene glycol), amines (such as urea), and an acid (such as oxalic acid or lactic acid). The present invention can use renewable DESs with lignin-derived phenols as HBDs. Both phenolic monomers and phenol mixture readily form DES upon heating at 100° C. with specific molar ratio with choline chloride. This class of DES does not require a multistep synthesis. The DES is synthesized from lignin which is a renewable source.

Both monomeric phenols and phenol mixture can be used to prepare DES. DES is capable of dissolving biomass or lignin, and can be utilized in biomass pretreatment and other applications. Using DES produced from biomass could lower the cost of biomass processing and enable greener routes for a variety of industrially relevant processes.

The DES, or mixture thereof, is bio-compatible: meaning the DES, or mixture thereof, does not reduce or does not significantly reduce the enzymatic activity of the enzyme, and/or is not toxic, and/or does not reduce or significantly reduce, the growth of the microbe. A “significant” reduction is a reduction to 70, 80, 90, or 95% or less of the enzyme's enzymatic activity and/or the microbe's growth (or doubling time), if the DES, or mixture thereof, was not present.

In some embodiments, the DES, or mixture thereof, comprises a quaternary ammonium salt and/or glycerol. In some embodiments, the DES, or mixture thereof, comprises a quaternary ammonium salt and/or glycerol. In some embodiments, the quaternary ammonium salt and/or glycerol have a molar ratio of about 1:1 to about 1:3. In some embodiments, the quaternary ammonium salt and/or glycerol have a molar ratio of about 1:1.5 to about 1:2.5. In some embodiments, the quaternary ammonium salt and/or glycerol have a molar ratio of about 1:1.8 or 1:1.9 to about 1:2.1 or 1:2.2. In some embodiments, the quaternary ammonium salt and/or glycerol have a molar ratio of about 1:2. In some embodiments, the quaternary ammonium salt is a choline halide, such choline chloride.

In some embodiments, the DES is distillable if the DES can be recovered at least equal to or more than about 50%, 55%, 60%, 65%, 70%, 75%, 80%, or 85% yield by distilling over vacuum at a temperature at about 100° C., 110° C., 120° C., 130° C., 140° C., 150° C., or 160° C., or any temperature between any two of the preceding temperatures.

In some embodiments, the DES can be one taught in WO 2018/204424 (Seema Singh et al.), which is hereby incorporated in its entirety by reference.

In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the introducing step(s) produce a sugar and a lignin from the biomass. The lignin can further be processed to produce a DES. The sugar is used for growth by the microbe.

In some embodiments, the solubilizing is full, near full (such as at least about 70, 80, or 90%), or partial (such as at least about 10, 20, 30, 40, 50, or 60%). In some embodiments, the one-pot composition is a slurry. When the steps described herein are continuous, the one-pot composition is in a steady state.

In some embodiments, the introducing step can further comprise heating the mixture comprises increasing the temperature of the solution to a value within a range of about 75° C. to about 125° C. In some embodiments, the heating step comprises increasing the temperature of the solution to a value within a range of about 80° C. to about 120° C. In some embodiments, the heating step comprises increasing the temperature of the solution to a value within a range of about 90° C. to about 110° C. In some embodiments, the heating step comprises increasing the temperature of the solution to about 100° C.

### Enzyme

In some embodiments, the enzyme is a cellulase. In some embodiments, the enzyme is thermophilic or hyperthermophilic. In some embodiments, the enzyme is any enzyme taught in U.S. Pat. Nos. 9,322,042; 9,376,728; 9,624,482; 9,725,749; 9,803,182; and 9,862,982; and PCT International Patent Application Nos. PCT/US2015/000320, PCT/US2016/063198, PCT/US2017/036438, PCT/US2010/032320, and PCT/US2012/036007 (all of which are incorporated in their entireties by reference).

### Microbe

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Generally, although not necessarily, the microbe is a yeast or a bacterium. In some embodiments, the microbe is Rhodosporidium toruloides or Pseudomonas putida. In some embodiments, the microbe is a Gram negative bacterium. In some embodiments, the microbe is of the phylum Proteobacteria. In some embodiments, the microbe is of the class Gammaproteobacteria. In some embodiments, the microbe is of the order Enterobacteriales. In some embodiments, the microbe is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. Suitable eukaryotic microbes include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments the microbe is a bacterium. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. lichenformis, B. anthracis, B. amyloliquefaciens, or B. pumilus.

### Biofuel

In some embodiments, the biofuel produced is ethanol, or any other organic molecule, described produced in a cell taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

### Biomass

The biomass can be obtained from one or more feedstock, such as softwood feedstock, hardwood feedstock, grass feedstock, and/or agricultural feedstock, or a mixture thereof.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picea rubens, Picea sitchensis, Picea glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus taeda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus taeda); spruce; and combinations/hybrids thereof.

Hardwood feedstocks include, but are not limited to, Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubing a; Buckeye (e.g. Aesculus hippocastanum, Aesculus glabra, Aesculus flava/Aesculus octandra); Butternut; Catalpa; Chemy (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hornbeam; Hophornbeam; Ipê; Iroko; Ironwood (e.g. Bangkirai, Carpinus caroliniana, Casuarina equisetifolia, Choricbangarpia subargentea, spp., Eusideroxylon zwageri, Guajacum officinale, Guajacum sanctum, Hopea odorata, Ipe, Krugiodendronferreum, Lyonothamnus lyonii (L. floribundus), Mesua ferrea, Olea spp., Olneya tesota, Ostrya virginiana, Parrotia persica, Tabebuia serratifolia); Jacarandi; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoume; Oregon Myrtle; California Bay Laurel; Pear; Poplar (e.g. P. balsamifera, P. nigra, Hybrid Poplar (Populus×canadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C₄ or C₃ grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, sorghum, rice, soybeans, hay, potatoes, cotton, or sugarcane. The feedstock can arise from the harvesting of crops from the following practices: intercropping, mixed intercropping, row cropping, relay cropping, and the like.

In some embodiments, the biomass is an ensiled biomass. In some embodiment, the biomass is ensiled by placing the biomass in an enclosed container or room, such as a silo, or by piling it in a heap covered by an airproof layer, such as a plastic film. The biomass undergoing the ensiling, known as the silage, goes through a bacterial fermentation process resulting in production of volatile fatty acids. In some embodiment, the ensiling comprises adding ensiling agents such as sugars, lactic acid or inoculants. In some embodiments, the ensiled biomass comprises one or more toxic compounds. In some embodiments, when ensiled biomass comprises one or more toxic compounds, the microbe is resistant to the one or more toxic compounds.

Refences cited herein:
1. Z. Usmani, M. Sharma, A. K. Awasthi, T. Lukk, M. G. Tuohy, L. Gong, P. Nguyen-Tri, A.
D. Goddard, R. M. Bill and S. C. Nayak, Renewable and sustainable energy reviews, 2021, 148, 111258.
2. N. Singh, R. R. Singhania, P. S. Nigam, C. D. Dong, A. K. Patel and M. Puri, Bioresource Technology, 2022, 344, 126415.
3. G. De Bhowmick, A. K. Sarmah and R. Sen, Bioresource technology, 2018, 247, 1144-1154.
4. B. Long, F. Zhang, S. Y. Dai, M. Foston, Y. J. Tang and J. S. Yuan, Nature Reviews Bioengineering, 2024, 1-15.
5. L. da Costa Sousa, S. P. Chundawat, V. Balan and B. E. Dale, Current opinion in biotechnology, 2009, 20, 339-347.
6. B. Kumar, N. Bhardwaj, K. Agrawal, V. Chaturvedi and P. Verma, Fuel processing technology, 2020, 199, 106244.
7. P. Kumar, D. M. Barrett, M. J. Delwiche and P. Stroeve, Industrial & engineering chemistry research, 2009, 48, 3713-3729.
8. M. Galbe and O. Wallberg, Biotechnology for biofuels, 2019, 12, 294.
9. S. Singh, G. Cheng, N. Sathitsuksanoh, D. Wu, P. Varanasi, A. George, V. Balan, X. Gao, R. Kumar and B. E. Dale, Frontiers in Energy Research, 2015, 2, 62.
10. K. H. Kim and C. G. Yoo, Frontiers in Chemical Engineering, 2021, 3, 785709.
11. L. Xu, J. Zhang, Q. J. Zong, L. Wang, T. Xu, J. Gong, Z. H. Liu, B. Z. Li and Y. J. Yuan, Chemical Engineering Journal, 2022, 427, 130962.
12. E. C. Achinivu, M. Mohan, H. Choudhary, L. Das, K. Huang, H. D. Magurudeniya, V. R. Pidatala, A. George, B. A. Simmons and J. M. Gladden, Green Chemistry, 2021, 23, 7269-7289.
13. L. Qin, W. C. Li, J. Q. Zhu, J. N. Liang, B. Z. Li and Y. J. Yuan, Biotechnology for biofuels, 2015, 8, 1-15.
14. L. Xu, M. Cao, J. Zhou, Y. Pang, Z. Li, D. Yang, S. Y. Leu, H. Lou, X. Pan and X. Qiu, Nature Communications, 2024, 15, 734.
15. S. P. Chundawat, G. Bellesia, N. Uppugundla, L. da Costa Sousa, D. Gao, A. M. Cheh, U. P. Agarwal, C. M. Bianchetti, G. N. Phillips Jr and P. Langan, Journal of the American Chemical Society, 2011, 133, 11163-11174.
16. L. Shuai and J. Luterbacher, ChemSusChem, 2016, 9, 133-155.
17. M. Mohan, N. Kumar, V. V. Goud, B. A. Simmons, K. L. Sale, J. M. Gladden, S. Singh and T. Banerjee, Fluid Phase Equilibr, 2022, 562, 113559.
18. M. Mohan, P. Viswanath, T. Banerjee and V. V. Goud, Mol Phys, 2018, 116, 2108-2128.
19. M. Mohan, B. A. Simmons, K. L. Sale and S. Singh, Scientific Reports, 2023, 13, 271.
20. M. Mohan, K. Huang, V. R. Pidatala, B. A. Simmons, S. Singh, K. L. Sale and J. M. Gladden,
Green Chemistry, 2022, 24, 1165-1176.
21. H. Liu, K. L. Sale, B. A. Simmons and S. Singh, The Journal of Physical Chemistry B, 2011, 115, 10251-10258.
22. H. Liu, G. Cheng, M. Kent, V. Stavila, B. A. Simmons, K. L. Sale and S. Singh, The Journal of Physical Chemistry B, 2012, 116, 8131-8138.
23. B. R. Taylor, N. Kumar, D. K. Mishra, B. A. Simmons, H. Choudhary and K. L. Sale, Molecules, 2024, 29, 5073.
24. N. Kumar, P. K. Naik and T. Banerjee, The Journal of Physical Chemistry B, 2022, 126, 4925-4938.
25. E. C. Achinivu, S. Frank, N. R. Baral, L. Das, M. Mohan, P. Otoupal, E. Shabir, S. Utan, C. D. Scown and B. A. Simmons, Green Chemistry, 2021, 23, 8611-8631.
26. S. Ntakirutimana, T. Xu, H. Liu, J. Q. Cui, Q. J. Zong, Z. H. Liu, B. Z. Li and Y. J. Yuan, Green Chemistry, 2022, 24, 5460-5478.
27. A. Casas, J. Palomar, M. V. Alonso, M. Oliet, S. Omar and F. Rodriguez, Industrial Crops and Products, 2012, 37, 155-163.
28. A. Casas, S. Omar, J. Palomar, M. Oliet, M. V. Alonso and F. Rodriguez, RSC advances, 2013, 3, 3453-3460.
29. N. Kumar, M. Mohan, J. C. Smith, B. A. Simmons, S. Singh and T. Banerjee, Journal of Molecular Liquids, 2024, 400, 124471.
30. K. A. Kurnia, S. o. P. Pinho and J. o. A. Coutinho, Industrial & Engineering Chemistry Research, 2014, 53, 12466-12475.
31. A. Klamt, The Journal of Physical Chemistry, 1995, 99, 2224-2235.
32. A. Klamt and F. Eckert, Fluid Phase Equilibr, 2000, 172, 43-72.
33. F. Eckert and A. Klamt, AIChE Journal, 2002, 48, 369-385.
34. J. Reinisch, A. Klamt, F. Eckert and M. Diedenhofen, Fluid Phase Equilibr, 2011, 310, 7-10.
35. W. Humphrey, A. Dalke and K. Schulten, Journal of molecular graphics, 1996, 14, 33-38.
36. T. Lu and F. Chen, Journal of computational chemistry, 2012, 33, 580-592.
37. D. Klemm, B. Heublein, H. P. Fink and A. Bohn, Angewandte chemie international edition, 2005, 44, 3358-3393.
38. C. Olsson and G. Westman, Cellulose-fundamental aspects, 2013, 10, 52144.
39. S. Köhler and T. Heinze, Macromolecular Bioscience, 2007, 7, 307-314.
40. Y. Chu, X. Zhang, M. Hillestad and X. He, Fluid Phase Equilibr, 2018, 475, 25-36.
41. P. Yamin, A. Bardow, K. O. Leonhard and I. Smirnova, COSMO-RS-based methods for improved modelling of complex chemical systems, Lehrstuhl für Technische Thermodynamik und Institut für Thermodynamik, 2019.
42. A. Tolbert, H. Akinosho, R. Khunsupat, A. K. Naskar and A. J. Ragauskas, Biofuels, Bioproducts and Biorefining, 2014, 8, 836-856.
43. R. Vanholme, K. Morreel, C. Darrah, P. Oyarce, J. H. Grabber, J. Ralph and W. Boerjan, New Phytologist, 2012, 196, 978-1000.
44. C. Balaji, T. Banerjee and V. V. Goud, Journal of solution chemistry, 2012, 41, 1610-1630.
45. P. Sannigrahi, A. J. Ragauskas and G. A. Tuskan, Biofuels, Bioproducts and Biorefining, 2010, 4, 209-226.
46. H. V. Scheller and P. Ulvskov, Annual review of plant biology, 2010, 61, 263-289.
47. D. Tarasov, M. Leitch and P. Fatehi, Biotechnology for biofuels, 2018, 11, 1-28.
48. M. Mohan, C. Balaji, V. V. Goud and T. Banerjee, Journal of Solution Chemistry, 2015, 44, 538-557.
49. A. Sluiter, B. Hames, R. Ruiz, C. Scarlata, J. Sluiter, D. Templeton and D. Crocker, National Renewable Energy Laboratory, 2008.
50. A. Yao, H. Choudhary, M. Mohan, A. Rodriguez, H. Magurudeniya, J. G. Pelton, A. George, B. A. Simmons and J. M. Gladden, ACS Sustainable Chemistry & Engineering, 2021, 9, 4371-4376.
51. R. S. Fukushima, M. S. Kerley, M. H. Ramos, J. H. Porter and R. L. Kallenbach, Animal Feed Science and Technology, 2015, 201, 25-37.
52. L. Segal, J. J. Creely, A. Martin Jr and C. Conrad, Textile research journal, 1959, 29, 786-794.
53. S. Park, J. O. Baker, M. E. Himmel, P. A. Parilla and D. K. Johnson, Biotechnology for biofuels, 2010, 3, 1-10.
54. J. Sameni, S. Krigstin and M. Sain, BioResources, 2017, 12, 1548-1565.
55. R. Wikandari, R. Millati and M. J. Taherzadeh, in Biomass fractionation technologies for a lignocellulosic feedstock based biorefinery, Elsevier, 2016, pp. 255-280.
56. E. R. Johnson, S. Keinan, P. Mori-Sinchez, J. Contreras-Garcia, A. J. Cohen and W. Yang, Journal of the American Chemical Society, 2010, 132, 6498-6506.
57. B. A. Marekha, O. N. Kalugin and A. Idrissi, Physical Chemistry Chemical Physics, 2015, 17, 16846-16857.
58. G. Cheng, P. Varanasi, R. Arora, V. Stavila, B. A. Simmons, M. S. Kent and S. Singh, The Journal of Physical Chemistry B, 2012, 116, 10049-10054.
59. H. Choudhary, B. A. Simmons and J. M. Gladden, Frontiers in Energy Research, 2022, 10, 868181.
60. A. D. French and M. Santiago Cintrón, Cellulose, 2013, 20, 583-588.
61. Y. Pu, F. Hu, F. Huang, B. H. Davison and A. J. Ragauskas, Biotechnology for biofuels, 2013, 6, 1-13.
62. M. Hall, P. Bansal, J. H. Lee, M. J. Realff and A. S. Bommarius, The FEBS journal, 2010, 277, 1571-1582.

### Example 1

### Multi-Scale Computational Screening and Mechanistic Insights of Cyclic Amines as Solvent for Improved Lignocellulosic Biomass Processing

Lignocellulose is a very promising bioresource for driving the development of sustainable processes that are based on renewable resources. Effective deconstruction of each biomass fraction is essential for viable biorefineries in the future. The present study computationally identifies solvents that can efficiently dissolve selective fractions of biopolymers and therefore can be used to extract them from lignocellulosic biomass during pretreatment, a process known to reduce recalcitrance to enzymatic deconstruction and increase conversion efficiency. The potential of 650 cyclic amines to solubilize lignin, cellulose, and hemicellulose was assessed based on the activity coefficients of solvent/biopolymers predicted by COSMO-RS (COnductor-like Screening MOdel for Real Solvents). The screening predicted that 1-piperazineethanamine (PzEtN) was likely a very effective solvent for selective lignin extraction and sequentially promote the highest lignin removal (97.1%) experimentally. To elucidate the dissolution mechanism of lignin, cellulose, and hemicellulose by these solvents, Non-Covalent Interaction(NCI)-Reduced Density Gradient(RDG) and quantum chemical calculations(interaction energies) were conducted, indicating that PzEtN and 1-methylimidazole (MeIm) demonstrate robust electrostatic interactions and hydrogen bonding with lignin, which lead to enhanced lignin removal while strong intramolecular hydrogen bonding interaction dominates in cellulose and hemicellulose result in less accessible for the solvents. Overall, these computational techniques offer an efficient method for quickly identifying solvents tailored for optimal biomass pretreatment.

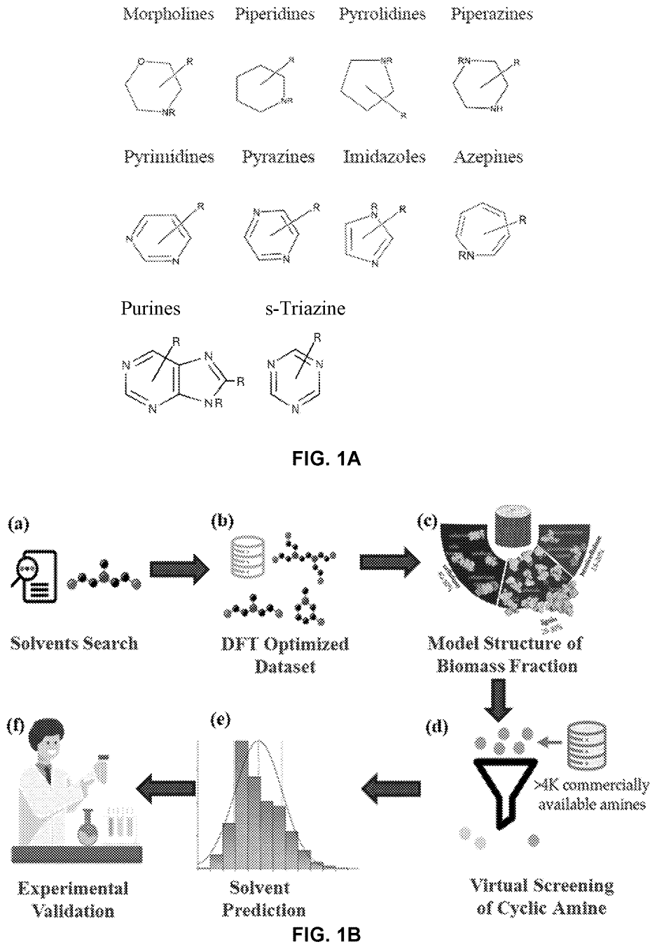

Recent work with amines^{12, 14, 25, 26} as pretreatment solvents has established key advantages, including high solids loading, effective biomass fractionation and delignification, and ease of separation. These studies suggested a strong influence of nucleophilicity and basicity on pretreatment effectiveness, while also stressing the stability issues related to primary amines. Continuing our research efforts on amines as pretreatment solvents, herein we further explore the suitability of a particular class of amines, i.e., cyclic amines, an underutilized component of the amine toolbox, in lignocellulosic biomass processing. Cyclic amines, secondary or tertiary amines with one or more nitrogen atoms, are attractive due to their high nucleophilicity and basicity, higher stability, and availability (lower cost) compared to primary amines. This study provides a thorough computational screening of over 650 unexplored cyclic amine solvent candidates to identify specific solvents that can help to dissolve either a specific biomass component or all components simultaneously (see FIG. 1A for various classes of cyclic amines including aromatic employed in this study). Ultimately, to achieve a more profound mechanistic comprehension of the solvents' role in biomass fraction dissolution, quantum chemical simulations are conducted to examine the interactions between the solvent and lignin, cellulose, and hemicellulose. The non-covalent interaction (NCI) and reduced density gradient (RDG) analyses were conducted to examine the strength and characteristics of hydrogen bonding in lignin and molecular solvents. This investigation revealed critical insights into lignin dissolution, demonstrating that hydrogen bonding between the solvent and lignin is a primary factor influencing biomass breakdown. The computational findings were experimentally validated for two down selected amines. The COSMO-RS prediction, coupled with mechanistic insights into biopolymer dissolution, establishes a robust basis for swiftly discovering efficient solvents for biomass pretreatment and advancing economical lignocellulosic conversion technologies.

### Methods

### 2.1 Computational Methods

COnductor-like Screening MOdel for Real Solvents (COSMO-RS) calculations were carried out to measure the capability of each cyclic amine to solubilize lignocellulosic biomass fractions based on the computed logarithmic activity coefficients. The ln(γ) values are often used as a quantitative descriptor for the dissolution power of a solvent. In the literature, ln(γ) has been reported as the dominating parameter in deciding the capability of a solvent and has also been successfully employed in previous studies to predict the solubility of cellulose and lignin in solvents.^{12, 27, 28} The Turbomole program (TmoleX version 23.0.0) was used to build the structural configurations of individual molecules. Density functional theory (DFT) modeling was performed utilizing the “TZVP-def” set with self-consistent field convergence at 1×10⁻⁶ Hartree within 500 cycles. Specifically, the Merck molecular force field (MMFF) was used as the basis for RDKIT, a random distance geometry algorithm, which TmoleX automatically employs by default to generate an initial geometry. Next, density-functional theory (DFT) was used to optimize the geometry of each of the cyclic amines and biopolymers by estimating the low-energy conformers, and then cosmo files were created.

Cosmo files were generated using the BIOVIA COSMOtherm software (2023 edition), and σ-profiles were subsequently generated using the BP_TZVP_23.ctd parameterization to calculate the thermophysical properties. The resulting σ-profiles can be replicated by utilizing the identical software, basis set, and DFT parameterization, specifically the def2-TZVP basis and B3-LYP functional with DFT-D3(BJ) dispersion correction in TmoleX. We performed a search for conformations of biomolecules and amines using the Turbomole and BIOVIA COSMOconfX2023 program (version 23.0.0, BIOVIA, Germany), which automatically identifies conformers for subsequent COSMO-RS calculations. COSMO calculations in COSMOConf were performed using the BP-TZVP method and basis set. FIG. 1B shows the workflow of the proposed QM-based prediction methodology and latter experimental validation.

The logarithmic activity coefficient, ln(γ), of component i is related to the chemical potential as given in the following equation 1.^{29, 30}

$$ln {γ}_{i} = \frac{{μ}_{i} - {μ}_{i}^{o}}{RT} \qquad ( 1 )$$

where,

$${μ}_{i}^{o}$$

is the chemical potential of the pure component i, R and Tare the ideal gas constant and absolute temperature. Additional details on the methodology of COSMO-RS calculations in predicting activity coefficients are provided elsewhere.³¹⁻³⁴

### 2.2 Quantum Chemical Calculations

In addition to COMO-RS calculations, quantum chemical (QC) calculations were performed to understand the mechanistic behavior of biomass fraction dissolution in the investigated cyclic amine solvents. For QC simulations, the complex structures of different biopolymers and cyclic amines were drawn in TmoleX. The geometries of the complex molecular systems were fully optimized at the specific def2-TZVP basis and B3-LYP functional with DFT-D3(BJ) dispersion correction in TmoleX including different conformers.

From QC calculations, the interaction energy (ΔE_{Interaction Energy}) is calculated by equation 2

$$Δ {E}_{Interaction Energy} ( kcal {mol}^{- 1} ) = {E}_{complex} - ( \sum {E}_{individual molecules} ) \qquad ( 2 )$$

where, E_{complex} is the total energy of the complex system (i.e., biopolymer+solvent) in kcal mol⁻¹. E_{Individual molecules} are the energies of the individual biopolymer or protic solvent in kcal mol⁻¹. Further, to examine the nature of intermolecular interactions in the biopolymer plus solvent systems, reduced density gradient non-covalent interactions (RDG-NCI) were analyzed using Multiwfn and VMD packages.^{35, 36}

### 2.3 Modeling of Representative Biomass Fractions

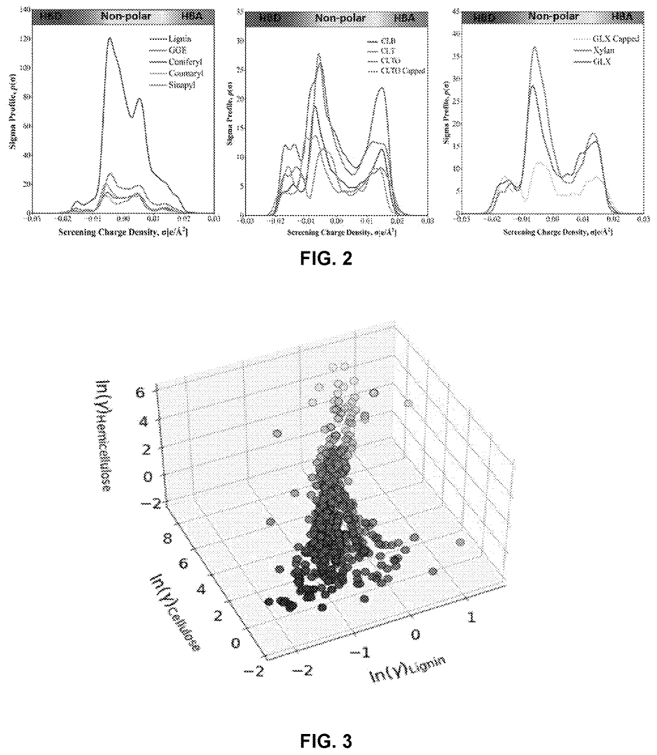

To predict the interaction of biomass components with cyclic amines as solvents, it is essential to first model these biomolecules with the continuum solvation model. The amounts of each component of the lignocellulosic biomass and the structure of the macromolecules may change based on the biological source. Lignocellulosic biopolymers generally exhibit substantial molar weight (>150 kDa for cellulose, >30 kDa for hemicelluloses, and ~2-15 kDa for lignin); thus, simulating their native structures is computationally challenging. To save computational time and complexity, the biomass fractions in this study were modeled separately as components of the original polymeric structure, as illustrated in FIG. 2. We modeled four structures as prospective representatives of the cellulose fraction, evaluated five structures for their capacity to represent the lignin fraction, and developed three model molecules for the hemicellulose fraction. The structures of all representative biomolecules as shown in FIG. S1 generated using the COSMO-RS model are included in the ESI and were modeled at a quantum chemical level as explained in the computational method of Section 2.1.

### Cellulose

The primary component of wood is cellulose, which is composed of polymers of glucose monomers that are β-1-4-glycosidically connected and have a degree of polymerization of up to 10,000 units.³⁷ These polymers are hydrogen bonded together to form crystalline cellulose. Cellulose is insoluble or only slightly soluble in the majority of common organic solvents³⁸ because of its chemical structure and strong intra- and intermolecular hydrogen bonding. Although DMSO-tetrabutylammonium fluoride, N-methyl morpholine oxide (NMMO), and ILs are solvents that are known to be able to solubilize cellulose to a considerable degree, they are either poisonous, thermally unstable, costly, and/or challenging to recycle.³⁹ In the literature, various molecules of cellulose have been delineated. Casas et al. employed a single glucose monomer in the most basic scenario for screening of ILs using the COMSO-RS model.²⁷ Nevertheless, the solubilization properties of glucose are significantly different from those of cellulose. Chu et al. used glucose, cellobiose, cellotriose, and cellotetraose as typical molecules to predict the excess enthalpies of cellulose in ILs.⁴⁰ The predicted excess enthalpies of cellobiose and cellotetraose had the strongest match with experimental solubility data in their investigation. A prevalent method for managing polymers using COSMO-RS involves doing quantum mechanical calculations on a computationally viable biopolymer fragment, followed by the truncation of its terminal groups. The entire polymer fragment undergoes geometric optimization, with mid-groups positioned as if integrated inside the polymer chain. The impact of the end-groups is typically minimal in the native polymer structure, necessitating their removal for solubility estimates. Both investigations emphasized the significance of intramolecular hydrogen bonding, which varies among different conformers of molecular cellulose. Yamin et al. asserts that hydrogen bonding is more accurately represented when two mid-monomers, as in cellotetraose, are considered rather than a single mid-monomer, as in a truncated cellotriose molecule.⁴¹ Utilizing this knowledge, we conducted a conformer search for all modeled biomolecules and evaluated cellobiose, cellotriose, cellotetraose, and a capped cellotetraose molecule for their efficacy as cellulose representations in interactions predictions.

### Lignin

Most lignin has a molar mass between 2,500 and 15,000 Da and is an amorphous polymer composed of three main phenylpropanoid units p-coumaryl, coniferyl, and sinapyl monolignols bonded together via radical coupling reactions. Subunits of lignin include p-hydroxyphenyl(H), guaiacyl(G), and syringyl(S).⁴² The quantity of each subunit varies considerably based on the biomass source. Lignin derived from herbaceous biomass consists of all three subunits. Softwood lignin has mostly G units (>95%), while hardwood lignin consists of S and G units predominantly (H content is typically <8%).⁴³ The connections between lignin subunits primarily consist of ether motifs and carbon-carbon bonds. The β-O-4 ether linkage is the predominant bond type joining monolignols and is also the most readily cleaved, but there are substantial quantities of other intricate connections that make lignin a challenging target for solubilization. Prior COSMO-RS investigations predominantly utilized monolignols as representatives of lignin.⁴⁴ Casas et al. employed pinoresinol and guaiacyl glycerol-2-coniferyl ether with monolignols as typical lignin compounds.²⁸ Gladden and coworkers employed S- and G-units linked through all principal linking motifs for solubility predictions in different study.^{12, 20} In this study, we modeled S, G, and H, together with dimers and trimers of S- and G-units linked by β-O-4 bonds. Furthermore, we simulated a conformer of a 2,500 Da lignin fragment with DP=26 for solubility predictions utilizing COSMO-RS.

### Hemicellulose

Hemicelluloses are a diverse class of polysaccharides found in plant cell walls. Hemicellulose primarily contains xylose as a sugar, with smaller amounts of mannose, galactose, arabinose, and rhamnose present as well. An acetyl group or uronic acid is another typical chain addition^{45, 46} Degrees of polymerization are typically between 50 and 300 units. Amorphous and hydrophilic, hemicellulose dissolves quite easily in water at high temperatures (>150° C.). An exception to this is the lignin-carbohydrate complex (LCC) that exists in biomass when cellulose and hemicellulose are bound to lignin. Benzyl ether, ester, and phenyl glycosidic linkages are by far the most prevalent types of lignin-carbohydrate interactions. Due to its strong bonding, the LCC can partially degrade in alkaline environments and hinder the enzymatic hydrolysis of biomass.⁴⁷ A combination of xylose and glucose monomers was used to represent hemicellulose in recent research.^{18, 48} The hemicellulose fraction is represented in this investigation by glucuronoxylan, xylan, and glucuronoxylan capped which are primarily found in dicots.

### 2.4 Environmental Health and Safety and Boiling Point

The physicochemical properties and hazards data for the studied compounds were obtained from established chemical databases. Boiling point data was retrieved from ChemSpider, a free chemical structure database maintained by the Royal Society of Chemistry. For compounds with multiple reported boiling points, only values at standard pressure (1 atm) were considered, and these values were averaged to obtain a single representative boiling point for each compound. Environmental, health, and safety (EHS) hazard classifications were extracted from PubChem, the open chemistry database of the National Institutes of Health. The hazard categories collected include acute toxicity, corrosivity, environmental hazards, flammability, general health hazards, and irritant properties. These classifications are based on the Globally Harmonized System of Classification and Labeling of Chemicals (GHS). Binary indicators were used to denote the presence of each hazard classification for each compound in the dataset.

### 2.5 Experimental Methodology

### Materials

Sorghum (Sorghum bicolor) biomass was used throughout this work and was donated from Idaho National Laboratories (Idaho Falls, USA). The biomass was dried at 40° C. for 24 h prior to use and subsequently screened through a 2 mm screen (Thomas-Wiley Model 4, NJ, USA). The biomass was stored in a cool, dry place in a leak- and air-resistant container to avoid moisture, until further use. Cyclic amines were purchased from Sigma Aldrich (St. Louis, MO, USA), including 1-piperazineethanamine (1-(2-aminoethyl) piperazine) (99% purity), 1-methylimidazole (≥99% purity). Additionally, sugar glucose (≥99.5%), xylose (≥99%), sodium hydroxide, acetic acid (glacial), lignin (alkali, 96% purity), acetyl bromide (99% purity), ethanol (≥99%) were procured from Sigma-Aldrich, as well as sulphuric acid (72% and 95-98%) from VWR for high-performance liquid chromatography (HPLC) analysis.

### Pretreatment of Sorghum Biomass

In a typical experiment, 0.9 g of the biomass was mixed with the 5.1 g of solvent and loaded into a glass pressure tube (30 mL, Ace Glass Inc., Vineland, NJ, USA) and mixed well prior to the experiments. The pretreatment experiments were performed at 140° C. for 3 h of reaction time at a solid loading of 15 wt %. The pretreatment of biomass sorghum was carried out using a two-step process that involves early separation (or washing) to remove the solvent after pretreatment. Post-pretreatment, some amount of ethanol and water mixture (1:1) was added to the slurry to transfer the contents to a 50-mL Falcon tube and centrifuged at 4000 rpm to separate solids from liquids. Subsequently, the recovered solid was further washed with the same mixture to remove any residual cyclic amines. Finally, the recovered solid fractions were lyophilized before performing compositional analysis on the pretreated solids. All the experiments were performed in duplicate, and the average values were reported here. The solid recovery (% SR) after pretreatment was calculated based on equation 3.

$$\% SR = \frac{Weight of biomass recovered after lyophilization ( g )}{Weight of biomass used for pretreatment ( g )} \times 100 \qquad ( 3 )$$

### Compositional Analysis

The compositional analysis of pretreated and untreated biomass sorghum was performed to determine the glucan and xylan content following the modified two-step acid hydrolysis procedure previously described by NREL.⁴⁹ In summary, 10 mg of the dry biomass was used to which 100 μL of 72% w/w H₂SO₄ was added and incubated at 30° C. for 1 h. Subsequently, secondary hydrolysis was performed for all the samples at 4% w/w H₂SO₄ by diluting the acid in the samples using ultra-pure water at 121° C. for 1 h. After the two-step acid hydrolysis, the hydrolysates were filtered using 0.45 μm filter plates (Pall AcroPrep). Additionally, monomeric sugars (glucose and xylose) were determined by HPLC using an Agilent 1200 (Santa Clara, CA, USA) series instrument equipped with a refractive index detector and Bio-Rad Aminex HPX-87H column (Bio-Rad, Richmond, CA, USA), coupled with a guard column assembly. Product separation was obtained at 60° C. with 4 mM H₂SO₄ as a mobile phase at a flow rate of 0.6 mL min⁻¹.

For the lignin content, the acetyl bromide-based lignin assay method was employed as reported previously. 10 mg of untreated or pretreated biomass was weighed in a 2 mL glass screwcap vial. 1 mL 25% (v/v) acetyl bromide in glacial acetic acid was added to the vials containing biomass samples and incubated at 50° C. for 2 h with constant stirring at 300 rpm. After digestion, the sample was quickly cooled in an ice bath and then mixed with 60 μl of an acetic acid: 2 M NaOH:5 M hydroxylamine mixture (48:10:2) and 200 μL glacial acetic acid sufficient for complete solubilization of the lignin extract. Followed by centrifugation (3000 rpm for 5 min), the absorbance of the supernatant was measured at 280 nm. A standard curve was generated with alkali lignin (TCI America) subjected to identical treatment with acetyl bromide in acetic acid (Caution: Acetyl bromide must be operated in fume hood).^{50, 51}

### Structural Characterization Using P-XRD

The powder X-ray diffraction (P-XRD) data was collected on a PANalytical Empyrean X-ray diffractometer equipped with a PIXcel3D detector and operated at 40 kV and 30 mA using Cu Kα radiation (λ=1.5418 Å). The diffraction patterns were collected in the 20 range of 4-40° with a step size of 0.026° and an exposure time of 30 s. The crystallinity index was also calculated using the equation 4, where the ratio of the height of the 002 peak (1002) and the height of the minimum (Iₐₘ) between the 002 and the 101 peaks.^{52, 53}

$$\% CI = \frac{( {I}_{002} - {I}_{am} )}{{I}_{002}} \times 100 \qquad ( 4 )$$

where, I₀₀₂ is the intensity of the crystalline plane (002) and Iₐₘ is the minimum between (002) and (101) peaks and is at about 18°.

### RESULTS AND DISCUSSION

### 3.1 Sigma Profile of Lignocellulosic Biomass

The sigma (σ)-profiles of biomolecules provide information regarding their polarity, acidity, and basicity.³² The sigma profiles of the model compounds of biomass fractions (FIG. 2) were plotted and analyzed to determine the physicochemical properties of biomolecules. These profiles represent the probability p(σ) of a molecular surface segment having a specific screening charge density (SCD). The SCD refers to the distribution of charges on a molecule's surface, representing how the molecule interacts with its surrounding environment by creating a screening charge essentially as a measure of the molecule's polarity at different points on its surface. The interaction energies with other liquid-phase surface segments are determined by the screening charge density σ(e/Å²). The chemical potential and all subsequent thermodynamic predictions in COSMO-RS model theory are derived from the σ-profiles. The σ-profiles can be categorized as hydrogen bond donor (HBD) (−0.03 to −0.01 e/Å²), neutral (−0.01 to 0.01 e/Å²), and hydrogen bond acceptor (HBA) (0.01 to 0.03 e/Å²) regions.

As shown in FIG. 2(a), all lignin samples exhibit two peaks at −0.006 (the highest peak) and 0.005 e/Å², resulting from the slightly electropositive hydrogen atoms of aromatic rings and carbon atoms, respectively. Polymeric lignin has the largest peak at −0.006 e/Å² due to the aromatic ring's face. Oxygen atoms in β-O-4 bonds and unbound hydroxy groups cause HBA behavior, as depicted in FIG. 2(a) for all lignin samples. The hydrogen atoms of the unbound hydroxy groups cause a tiny peak in the HBD area. Thus, inter- and intramolecular hydrogen bonding is feasible, albeit less intense than with cellulose and hemicellulose. Lignin has broader neutral area peaks compared to cellulose and hemicellulose in its σ-profiles, suggesting different solubilization characteristics. All cellulose representatives (see FIG. 2(b)) exhibit strong HBA- and HBD-behavior, with peaks at 0.017 and −0.018 e/Å², respectively. There are chances for hydrogen bonds with possible solvents, and intramolecular hydrogen bonding is also possible. Additionally, cellulose exhibits a peak at −0.007 e/Å² due to sugar carbons in the neutral area. The peak height increases with chain length, notably in the neutral zone, indicating decreased solubility for polar solvents. Capped cellotetraose, which exclusively uses cellobiose as a repeating unit by inactivating the terminal ends. The repeating units of polymers deactivated as they represent similar behavior, and the handling of large polymers is computationally expensive. A solvent that dissolves cellulose should establish strong intermolecular hydrogen bonds and have neutral screening charges. Hemicellulose representatives (FIG. 2(c)) exhibit robust hydrogen bond formation due to their comparable σ-profiles to cellulose.

### 3.2 Comprehensive Dissolution of Lignin, Cellulose and Hemicellulose

Solvent selection for comprehensive dissolution of cellulose, lignin, and hemicellulose was a challenging task due to the complex nature of biomolecules. FIG. 3 shows ln(γ) values for all three fractions of biomass in the 650 cyclic amines at 140° C. and 1 atm pressure. The more negative value of ln(γ) indicates the higher solubility, signifying the solute is undergoing positive interaction with the solvent. The values reported in this work are calculated at infinite dilution unless otherwise specified. The solvents having the more negative value of ln(γ) for all three fractions of biomass are predicted as optimal solvents for combined dissolution of lignin, cellulose, and hemicellulose. There are several solvents that were identified as the optimal solvents for improved dissolution of lignin, cellulose and hemicellulose fraction of biomass.

In addition to the activity coefficient, the solvent screening process incorporated criteria for both physical properties and safety considerations. The boiling point range was constrained to 180-300° C., with the lower limit ensuring thermal stability during biomass processing and the upper limit facilitating energy-efficient solvent recovery and recycling. Environmental, health, and safety (EHS) classifications were evaluated using the Globally Harmonized System (GHS) framework, which categorizes hazards into six primary types: acute toxicity, corrosivity, environmental hazards, flammability, general health hazards, and irritant properties. Solvents were assessed based on their hazard's profiles, with preference given to candidates showing fewer hazard classifications. While the complete absence of hazard markers was ideal, solvents with up to four hazard classifications were considered acceptable if they suggested superior biomass processing capabilities. Solvents exhibiting all six hazardous categories were automatically excluded from consideration. This systematic approach to solvent selection ensured that candidates not only met the technical requirements for effective biomass processing but also aligned with practical safety and handling considerations necessary for potential industrial implementation.

### 3.3 Best Solvents for Cellulose and Lignin

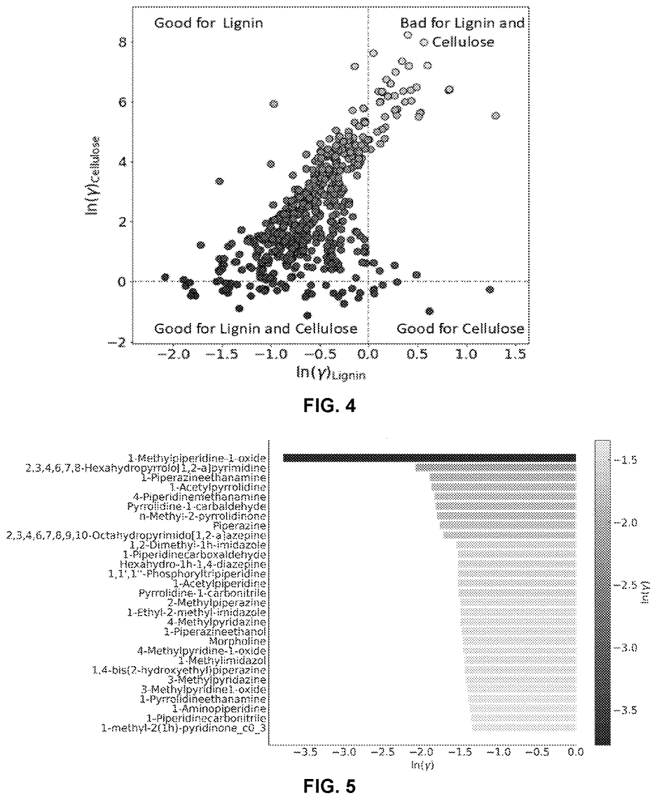

The solvent selection for joint dissolution of cellulose and lignin was challenging due to the entirely different behavior of lignin and cellulose. FIG. 4 shows the joint dissolution of lignin and cellulose for the cyclic amine's interactions at 140° C. and 1 atm pressure. The solvents having the more negative value of ln(γ) for lignin and cellulose are considered as the optimal solvents for combined dissolution. FIG. 4 is divided into four parts depending on the ln(γ) values for lignin and cellulose, which aids in identifying the solvents relative to their interaction with lignin and cellulose. Based on the plot data some of the solvents identified as best solvents for joint dissolution of lignin and cellulose, which were tested experimentally. To further understand the behavior of hemicellulose and cellulose with cyclic amine solvents, we plotted the lignin vs hemicellulose and cellulose vs hemicellulose at same operating conditions and shown in FIG. S1 and FIG. S2 respectively. The cellulose vs hemicellulose shows the straight-line relationship due to similar behaviors of monomeric unit towards the solvents as they are constituent of sugars.

### 3.4 Selective Lignin Extraction Using Cyclic Amines as Solvent

Multiple efforts have been made to identify appropriate solvents that can solubilize the lignin in the lignocellulosic biomass to enable efficient processing of cellulose. Ideally, an appropriate solvent would selectively dissolve lignin and not interact with the holocellulosic component of the lignocellulosic biomass. Consequently, for this argument, we designated the best point as low-cellulose, high-lignin solubility solvents. Solvents preferentially extract lignin, leaving cellulose as a readily separate solid, consistent with typical fractionation methods. A substantial array of known solvents facilitates the understanding of the structural patterns responsible for elevated lignin solubilities. We identified numerous structural similarities among the most promising solvent options from the list that aid in tuning solvents for lignin solvation. The detected solvents are categorized into the following principal classes: azines (including pyridines, pyrazines, pyrimidines, pyridazines, and triazines), and oxazolines. These solvents may possess superior solubility and more favorable EHS features, as shown for the top 30 candidates selected on the basis of selective lignin extraction in table S1. FIG. 5 shows the top 30 cyclic amines as solvents indicated for this purpose based on the activity coefficients calculated at 140° C. and 1 atm pressure.

Aniline and pyridine were recently reported as the solvents for lignin dissolution, all of which were rediscovered by the presented solvent screening framework.⁵⁴ In addition to the already established lignin solvents such as pyridine, we discovered commercially available azoles, such as thiazole or isoxazole. Thiazole is only slightly toxic (LD50 oral rat: 938 mg/kg), while toxicity data for isoxazole is not available. Further aromatic N-heterocycles were designed, including triazines, diazines, pyridines, bicyclic compounds, and aromatic compounds. Common side chain motifs were methoxy-, alkyl-, and NH₂-groups. Pyridines and many diazines have benign EHS properties and are readily commercially available. Most triazines are solid at room temperature, limiting their applicability for lignin upgrading. During the solvent design, functional groups associated with low lignin solubilities (e.g., alkanes) were gradually replaced by functional groups associated with higher lignin solubilities (e.g., aromatic N-atoms), leading to a gradually increasing mean lignin solubility of the population. We were unable to find any experimental data on lignocellulose processing using 1-methylpiperidine-1-oxide, and it was not commercially available though it was one of the best predicted solvents for the dissolution of cellulose and lignin. Despite its slightly high price and low thermal stability, 1-methylpiperidine-1-oxide is structurally similar to the solvent NMMO, which is known to have excellent cellulose dissolving properties.⁵⁵ Similarly, according to the activity coefficients, 1-piperazineethanamine (PzEtN) and 1-methylimidazole (MeIm) are one of the best solvents for selective lignin extraction however PzEtN shows the ln(γ) values positive for cellulose indicates low dissolution capacity. Following this selection process, the cyclic amines MeIm and PzEtN were selected for experimental validation via pretreatment of sorghum biomass. Experimental findings in section 3.8 validate prediction result, emphasizing the uniqueness of our results alignment as PzEtN showed the exceptionally higher lignin removal (97.1%) and lower cellulose dissolution. These predictions provide guidance for innovative QM based predictive framework utilizing newly identified solvents from the novel class of solvents, such as cyclic amine.

### 3.5 Sigma Potential Insights of Biopolymers and Cyclic Amines Interactions

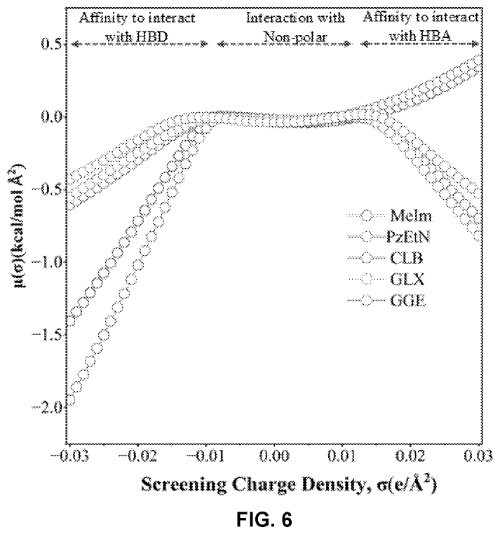

The sigma potentials (μ(σ)) of the biopolymer's cellulose, hemicellulose, lignin and the cyclic amines MeIm and PzEtN were calculated to understand the affinity of solvents for the surface polarity of different biomass fractions. The σ-potentials are divided into three main types: non-polar (0.01 e/Å²<σ>+0.01 e/Å²), H-bond acceptor (σ<−0.01 e/Å²), and H-bond donor (σ>+0.01 e/Å²) regions (see FIG. 6). The sigma potentials, μ(σ), of the Lignin dimer (GGE), Cellobiose (CLB), and Glucuronoxylan (GLX) surrogate compounds are negative in both the negative and positive charge density regions (σ<−0.01 e/Å² and σ>+0.01 e/Å²), indicating that biopolymers tend to interact with both negative and positive polar surfaces of solvent molecules (i.e., H-bond donors and acceptors in the solvent). On the negative screening charge densities side (σ>−0.01 e/Å²), the σ-potential value of PzEtN (−2.0 kcal/mol Δ²) is more negative than the MeIm σ-potential (−1.4 kcal/mol Δ²), implying the former has more affinity to interact with the positive surface charge density of biopolymers (i.e., H-bond donors) and have high bond basicity, both of which would predict higher lignin solubility compared to cellulose and hemicellulose. In contrast, the σ-potential values of MeIm and PzEtN are positive (0.3-0.4 kcal/mol Å²) in the region of positive screening charge densities (α>+0.01 e/Å²), which reflects that MeIm and PzEtN lack electron donor surfaces. Thus, the intramolecular interactions in MeIm and PzEtN are very weak, which would enable stronger interaction with the biopolymers. The PzEtN has a higher tendency to interact with negatively charged surfaces, thereby forming stronger electrostatic and hydrogen bonding with lignin compared to MeIm. As a result, the logarithmic activity coefficient for the PzEtN with lignin was higher than MeIm. The PzEtN has showed a very weak interaction with CLB and GLX due to their existing strong intramolecular Hydrogen bonding.

### 3.6 Understanding the Mechanistic Behavior of Lignocellulosic Biomass Dissolution

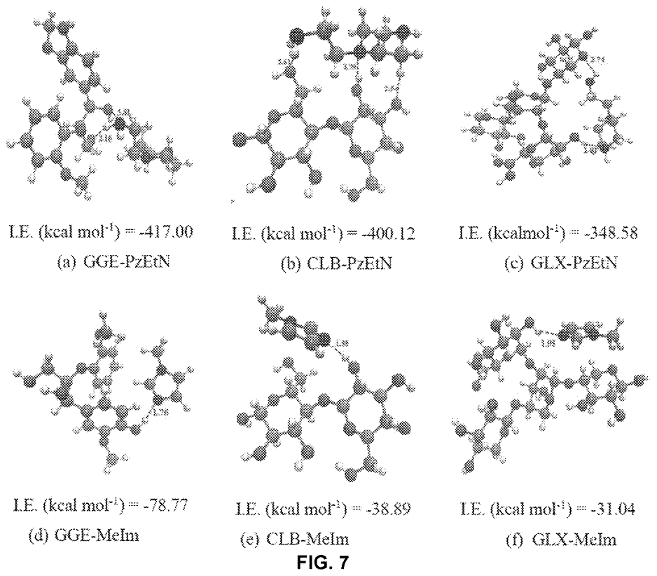

Quantum chemical (QC) simulations were performed to study how the cyclic amines as solvents interact with cellulose, hemicellulose, and lignin and to learn which interactions are critical for lignocellulosic biomass solvation. We selected guaiacyl glycerol-β-guaiacyl ether (GGE), a representative lignin dimer with the most common β-O-4 monomer-monomer linkage, as our model molecule for lignin because performing QC calculations with structures of large macromolecules like polymeric lignin is computationally expensive. Similarly, we used cellobiose (CLB) as the model compound for cellulose and glucuronoxylan (GLX) as hemicellulose to understand the solvation mechanism insights for different biomass fractions. The investigation of these (CLB, GLX, GGE) biopolymer/amine systems become more complicated due to their ability to adopt multiple conformations. The Boltzmann distribution states that the most abundant distribution is characterized by the conformers having the lowest energy, so the lowest energy conformer structures of the different biomass fractions were simulated with the two cyclic amines as solvents, PzEtN and MeIm, and used to gain mechanistic insights into biomass solvation. The biopolymer-solvent clusters and their relative interaction energies are provided in FIG. 7. The lignin dimer (GGE) in PzEtN had the highest interaction energy compared to cellulose and hemicellulose. The MeIm had ~5 times lower interaction energy with GGE compared to PzEtN. The interaction energy of GLX and CLB with PzEtN was ~10 times higher compared to MeIm, indicating that PzEtN interacts more strongly with all three biomass components. All the optimized conformers showed that the interaction site for the α-OH, 7-OH, and phenol-OH groups of GGE forms strong H-bonds with both cyclic amine solvents. Similarly, the amine functional group interacted with the hydroxyl group present in CLB, GLX and makes robust H-bonding, however cellulose and glucuronoxylan possess a strong intramolecular hydrogen bonding as depicted by NCI-RDG and sigma potential. As a result, it becomes difficult for the solvents to penetrate and reduce the crystallinity as corroborated by experimental finding in PXRD analysis. In addition, NCI-RDG analysis was performed for the most stable optimized conformers to dissect the non-covalent interactions responsible for solvation of the biopolymers in MeIm and PzEtN.

### 3.7 Reduced Density Gradient (RDG) Analysis of Biopolymers-Amine Solvents

Reduced Density Gradient (RDG) analysis was conducted to assess the strength of the non-covalent interactions (NCIs) between the CLB, GGE, GLX, and solvents, namely PzEtN and MeIm. This method can be used to visualize the different interaction energy contributions responsible for the dissolution of the different biomass fractions. The concept of RDG analysis is articulated using equation 5, which focuses on the study of electron density in distribution areas characterized by both low electron density(ρ) and its low gradient values.^{56, 57}

$$RDG = \frac{1}{2 {( 3 {π}^{2} )}^{\frac{1}{3}}} \frac{\vert ∇ ρ \vert}{{ρ}^{\frac{4}{3}}} \qquad ( 5 )$$

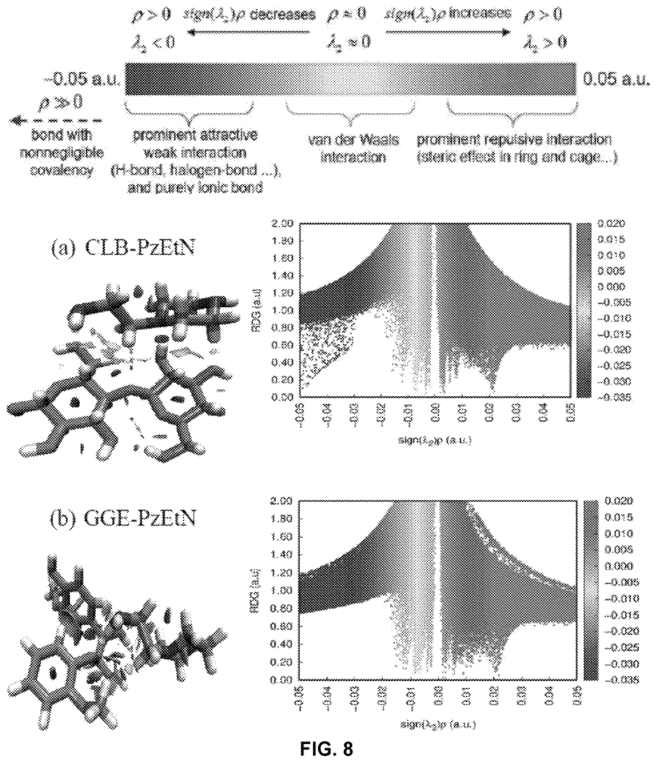

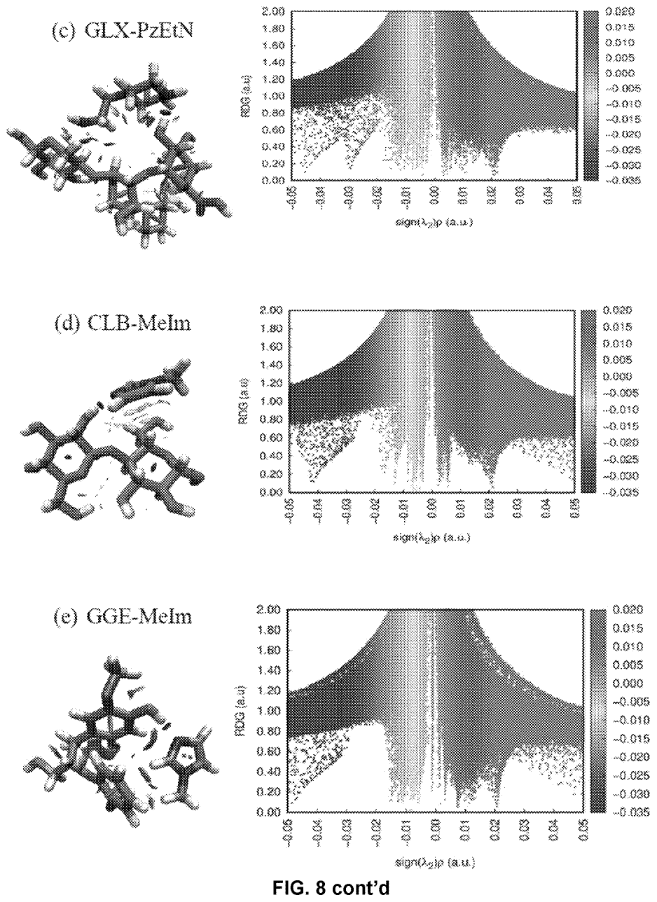

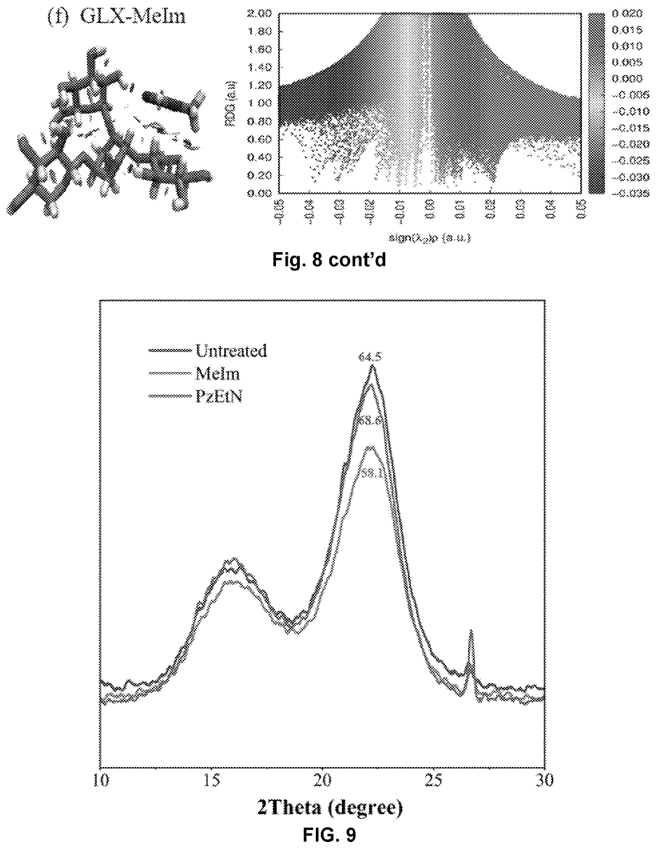

This equation sets the basis for NCI analysis, which interprets spikes in RDG as indicators of various weak non-covalent interactions. Utilizing RDG for NCI analysis can explain and quantify multiple weak interactions, such as H-bonds, vdW, and London dispersion interactions. The RDG scatter graph plotted against the electron density modified by the sign of the second eigen value of the electron density [sign(λ₂)ρ] unveils a spectrum of weak interactions. These non-covalent interactions are depicted through RDG isosurfaces and scatter plots, which display changes from positive to negative [sign(λ₂)ρ] values for different compounds, as shown in FIG. 8a-8f at using the def2-TZVP level of theory with D3BJ dispersion correction. Different colored peaks indicate different types of interactions. Blue areas(λ₂<0) are linked with strong, attractive forces like hydrogen/halogen bonds and electrostatic interactions, while red areas (λ₂>0) signify strong steric repulsion from nonbonded overlap. The intermediary or transition areas (λ₂≅0) are depicted in green and track van der Waals interactions, such as dispersion forces and dipole-dipole interactions.

The two cyclic amine solvents PzEtN and MeIm were explored in detail using this analysis (see FIGS. 8a-8f). Scanning across sign(λ₂)ρ(r) from positive to negative values, there are several spikes in the RDG scatter plot that correspond to the steric repulsion (red), van der Waal (green) interaction, and hydrogen bonding (blue). In FIG. 8a-8c, the interactions are visualized for PzEtN interaction with CLB, GGE, and GLX respectively. In general, if the RDG scatters in the range ±0.005, weak interactions such as dispersion force exist; if the RDG scatters in the range >0.005, strong repulsive interactions exist, such as steric hindrance in aromatic rings; and if the RDG scatters in the range <0.005, strong attractive interactions exist, such as hydrogen bonds. Examination of the NCI plots shows that the PzEtN have spikes in the negative region of sign(λ₂)ρ(r). The reason for extra, blue-colored spikes in CLB and GLX compared to GGE with PzEtN in the range of −0.05-0.03 was interpreted as being due to intramolecular hydrogen bonding between the complex structure of cellulose and hemicellulose. FIG. 8b shows the NCI-RDG plot of PzEtN-GGE, and it shows the minimal repulsive interaction compared to MeIm and no such intramolecular hydrogen bonding exists. The PzEtN interacts with GGE from the primary amine and interacts with the β-O-4 bond of lignin. On the other hand, in the attractive region, multiple spikes are observed for biopolymer fractions-cyclic amine interactions, which are consistent with the geometrical analysis either from sigma potential or interaction energy. In addition, certain repulsive interaction peaks in the range of 0.03-0.05 (red) (see FIG. 8d-8f) are more prevalent in the MeIm-biopolymers system, which can be attributed to the repulsive interaction of aromatic rings and is predicted to result in lower dissolution of biomass.

### 3.8 Experimental Validation: Pretreatment with Cyclic Amines

As described earlier, two cyclic amines with different predicted biomass solubilization properties, low cost, commercial availability, EHS, and boiling point MeIm and PzEtN, were selected to experimentally validate the results from the computational screening. To do so, pretreatment of sorghum biomass was performed, and the material obtained after the reaction was recovered and analyzed by measuring lignin, glucan, and xylan removal. Solids recovery after pretreatment was 66.8% for MeIm and 48.8% for PzEtN, indicating a stronger biomass solubilization effect by the latter under the tested conditions (15 wt. % solids, 140° C., 3 h). The residual biomass composition varied significantly between treatments in terms of lignin and cellulose content. When using PzEtN, we observed almost complete lignin removal (97.1%) with lower glucan losses (24.7%) and a slightly higher xylan removal (40.9%) (Table 1). Pretreatment with MeIm resulted in significantly lower lignin removal (57.5%) and higher cellulose losses (51.1%). This aligns with the expected interactions between amine solvents and lignocellulosic biopolymers. Furthermore, the near-complete lignin removal in the case of PzEtN results in a biomass with a more fragmented and porous texture when compared to a granular texture for the biomass pretreated with MeIm (FIG. S3). Since lignin extraction coincides with the removal of hemicellulose and cellulose, these results are consistent with typical behavior of biomass deconstruction. In a practical biorefinery setting, the extracted polysaccharides would be reintroduced into the saccharification reaction after solvent recovery.

TABLE 1
Compositional analysis of sorghum biomass before
and after pretreatment with two cyclic amines.

| | Solid | | | | | | |
|---|---|---|---|---|---|---|---|
| | recovery | Composition (%) | | | Removal (%) | | |
| | (%) | Glucan | Xylan | Lignin | Glucan | Xylan | Lignin |
| Untreated | | 39.1 ± 1.82 | 15.3 ± 0.1 | 28.4 ± 0.1 | | | |
| 1-Methyl | 66.8 ± 1.6 | 28.5 ± 2.4 | 16.1 ± 1.3 | 17.9 ± 0.1 | 51.1 ± 2.0 | 29.8 ± 1.1 | 57.8 ± 0.1 |
| Imidazole | | | | | | | |
| (MeIm) | | | | | | | |
| 1-Piperazineet | 48.8 ± 4.9 | 43.9 ± 1.2 | 18.6 ± 1.9 | 1.7 ± 0.1 | 24.7 ± 1.1 | 40.9 ± 1.6 | 97.1 ± 0.1 |
| hanamine | | | | | | | |
| (PzEtN) | | | | | | | |

We also investigated the structural changes that occurred to cellulose after pretreatment with the cyclic amines using powder X-ray diffraction. Specifically, we aimed to identify the cellulose polymorphs and the ratio of crystalline to amorphous components in the lignocellulosic biomass since conversion of crystalline cellulose to a more amorphous form is known to increase the efficiency of saccharification and yields of fermentable sugars.^{21, 22, 58} Furthermore, a change in the crystallinity index (CrI) is an indication of structural changes and depends on 1) the amount of crystalline cellulose dissolved and precipitated during biomass pretreatment and 2) the loss of non-crystalline components during washing of the pretreatment solvent.⁵⁹ The diffraction patterns observed in untreated sorghum correspond to the cellulose I polymorph, which is commonly associated with native cellulose found in untreated lignocellulosic biomass, with a CrI of 64.3%. When MeIm was used to pretreat the biomass, a lower CrI of 58.1% was obtained. Pretreatment with PzEtN resulted in a CrI of 63.6%. FIG. 9 shows the X-ray diffraction patterns of sorghum biomass before and after pretreatment with their CrI.

The contrasting effects of MeIm and PzEtN on lignin and cellulose reflect the importance of pretreatment choice for optimizing downstream processes. Pretreatments that decrease both lignin content and cellulose crystallinity tend to enhance enzyme accessibility, promoting more efficient cellulose hydrolysis. For instance, the moderate reduction in CrI and lignin content in the MeIm-treated sample depicts partial disruption of the cellulose structure due to pretreatment and result in broader and less intense peaks, which indicates a shift towards a more amorphous structure with granular and moderately broken-down texture (FIG. S3).^{60, 61} In contrast, for PzEtN pretreatment despite significant lignin removal (97.1%), the CrI remains close to that of untreated sorghum biomass. It suggests pretreatment effectively removes amorphous lignin (and hemicellulose to some extent) but does not significantly impact cellulose crystallinity. The sharpness of peak at 22° supports this interpretation, as the cellulose microfibrils remain largely intact. This corresponds to the loose, disrupted texture seen in the image indicating a delignified yet crystalline cellulose structure (FIG. S3). This outcome underscores the importance of balancing lignin removal with partial cellulose amorphization to maximize the saccharification yield.^{58, 62}

### CONCLUSION

The current work demonstrated an effective framework for 1) discovering and predicting high-performing solvents for the deconstruction of different lignocellulosic biomass fractions and 2) understanding the mechanistic factors that control the biopolymer dissolution capacity of a solvent. The study established a quantum mechanics-based solvent screening framework for the improved processing of lignocellulosic biomass, which was then experimentally verified. The computational approach employed rigorously evaluated representative biomolecules to predict the activity coefficient of biomass fractions using the COSMO-RS and sequentially it was later verified experimentally using 1-piperazineethanamine (PzEtN) and 1-methylimidazole (MeIm). The identification of unexplored cyclic amines that exhibited elevated lignin extraction (97.1%) was exceptionally higher when compared to other reported solvents. The lignin dimer (GGE) and PzEtN show the highest interaction energy compared to cellulose and hemicellulose, result in highest lignin removal. The MeIm shows ~5 times lower interaction energy with GGE compared to PzEtN, resulting in lower lignin removal. The QC methods presented aid in identifying the functional groups that determine biopolymer solvation, such as azines and oxazolines, and they can be tuned easily by replacing degree of branching, functional group, and ring structures. The PzEtN higher lignin removal may be more advantageous in lignin-first biorefinery approaches requiring almost complete delignification, although additional steps may be necessary to address the retained cellulose crystallinity. As a future perspective, the feasibility and sustainability of the identified solvents in biorefinery processes, including fractionation and downstream processing, should be assessed. To incorporate the identified solvents into a particular biorefinery process, it is necessary to conduct analyses of their stability, recovery, recycling, cost, and product selectivity.

### Example 2

### Thermodynamic Relationship Between Cellulose and Lignin

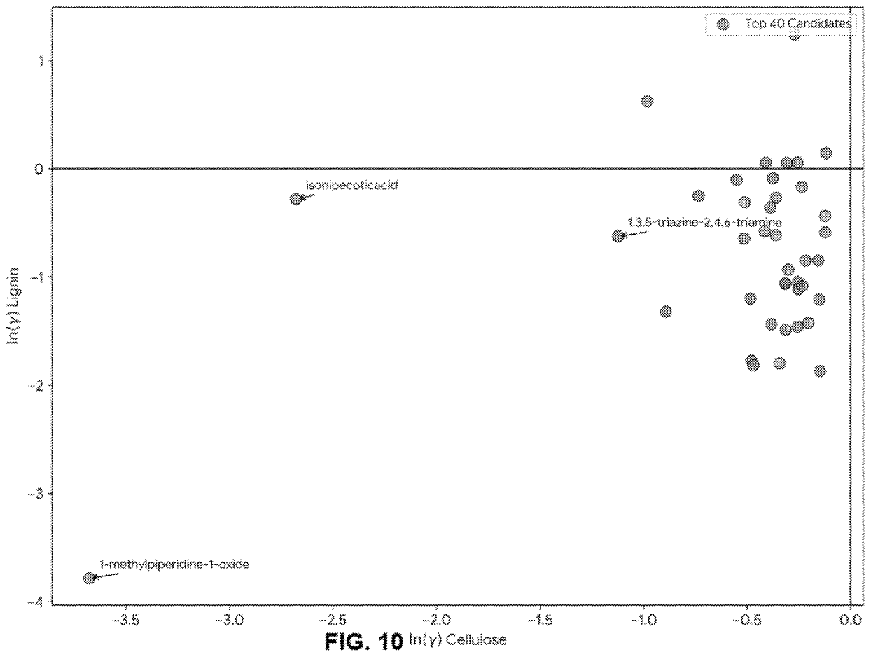

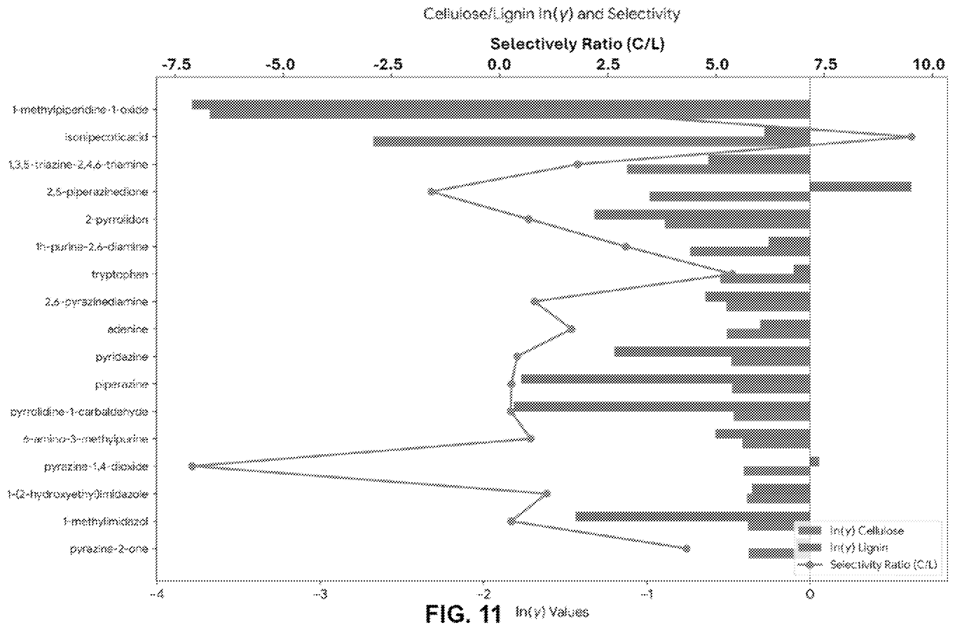

The scatter plot (FIG. 10) illustrates the thermodynamic relationship between cellulose and lignin for the top 40 candidate solvents, specifically those characterized by negative activity coefficients for cellulose (ln(γ)_{Cellulose}<0). By mapping ln(γ))_{Cellulose} against ln(γ)_{Lignin}, the visualization identifies solvents capable of selective biomass fractionation. Solvents positioned in the upper-left region, such as 1-methylpiperidine-1-oxide, isonipecoticacid, and 1,3,5-triazine-2,4,6-triamine, exhibit superior performance by maximizing cellulose solubility while minimizing lignin interaction. This is providing a clear benchmark for identifying efficient solvents that disrupt crystalline cellulose without co-dissolving lignin during lignocellulosic pretreatment with the solvents

This plot (FIG. 11) represents individual activity coefficients ln(γ), where more negative values signify higher solubility. Intersecting these bars is a green line representing the selectivity ratio

$$( \frac{{ln ( γ )}_{Cellulose}}{{ln ( γ )}_{Lignin}} )$$

offering a direct metric for fractionation efficiency. Solvents like ln(γ)Lignin 2,5-piperazinedione demonstrate a distinct “lignin-positive” profile, indicating they repel lignin while effectively dissolving cellulose. This combined representation allows researchers to simultaneously evaluate absolute solubility and relative selectivity, which is essential for optimizing chemical processes in sustainable biorefineries.

### Methodology

Pretreatment of sorghum and pine biomass was conducted in 30 mL glass pressure tubes (Ace Glass Inc., Vineland, NJ, USA). Prior to the experiments, 0.9 g of biomass was thoroughly mixed with 5.1 g of solvent. Pretreatment was performed at 40° C. overnight under the following conditions: (1) pine at 40° C., (2) sorghum with neat PzEtN at 40° C., and (3) sorghum with 80% PzEtN and 20% water (aqueous PzEtN) at 40° C.

Biomass pretreatment followed a two-step process involving initial separation by filtration and mechanical pressing to remove the solvent after treatment. The resulting slurry was then washed with 1:1 ethanol and water mixture and transferred into 50 mL Falcon tubes. Samples were centrifuged at 4000 rpm at 4° C. for 20 mins to separate solids and liquids. The recovered solids were further washed with the same ethanol and water mixture to ensure complete removal of residual cyclic amines, until a clear solvent with neutral pH (~7) was obtained. Finally, the solid fractions were lyophilized prior to compositional analysis.

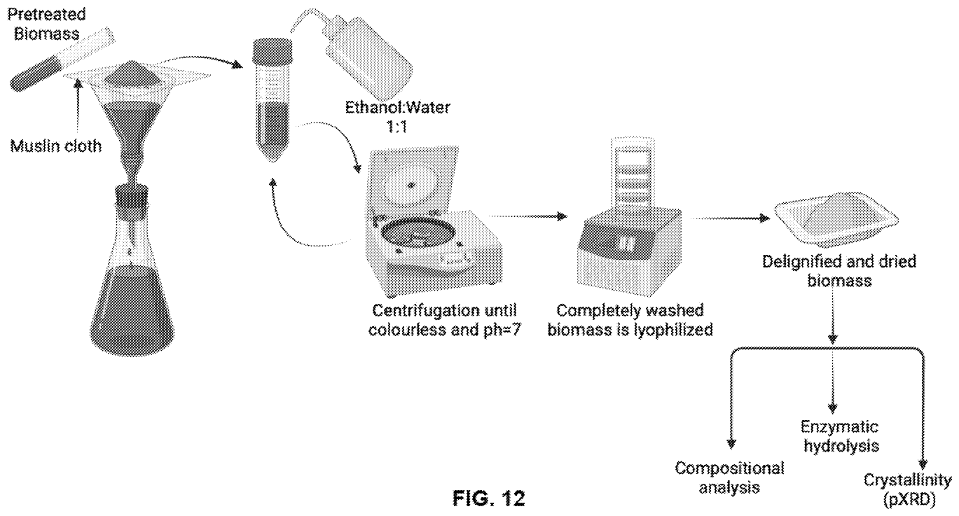

Lignin removal was more effective in sorghum, a representative grassy biomass, compared to pine, a softwood biomass, under pretreatment with piperazineethanamine at 40° C. Additionally, the use of an aqueous cyclic amine system resulted in slightly enhanced lignin removal and improved sugar conversion following enzymatic hydrolysis relative to neat PzEtN. Overall sugar yields, particularly glucose and xylose, remained significantly low for pine under these conditions. See FIG. 12.

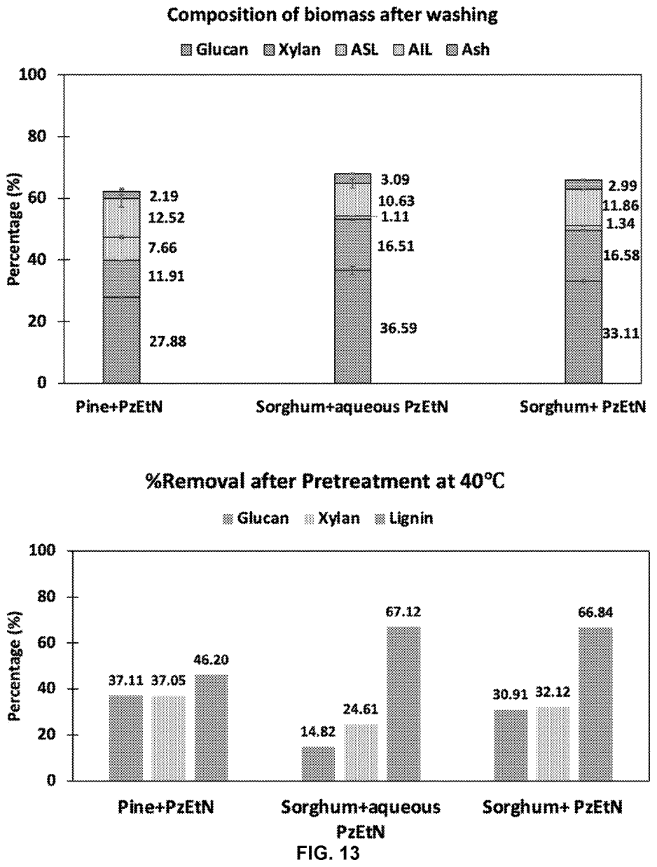

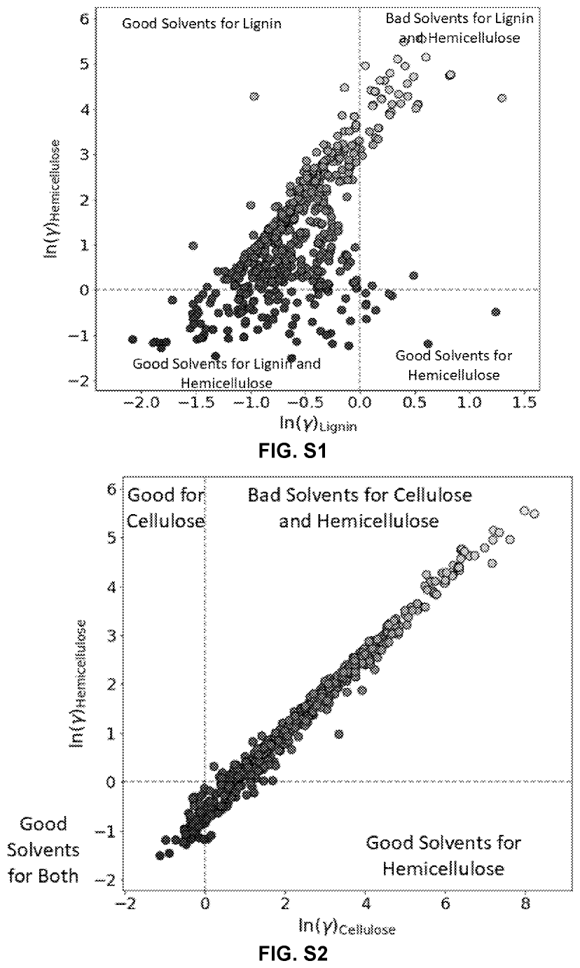

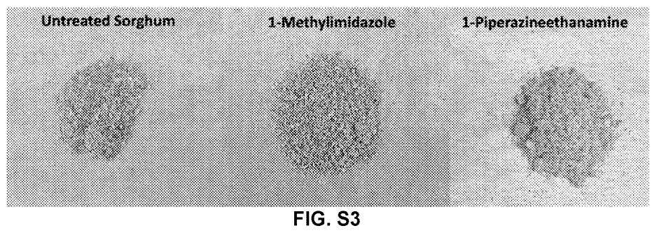

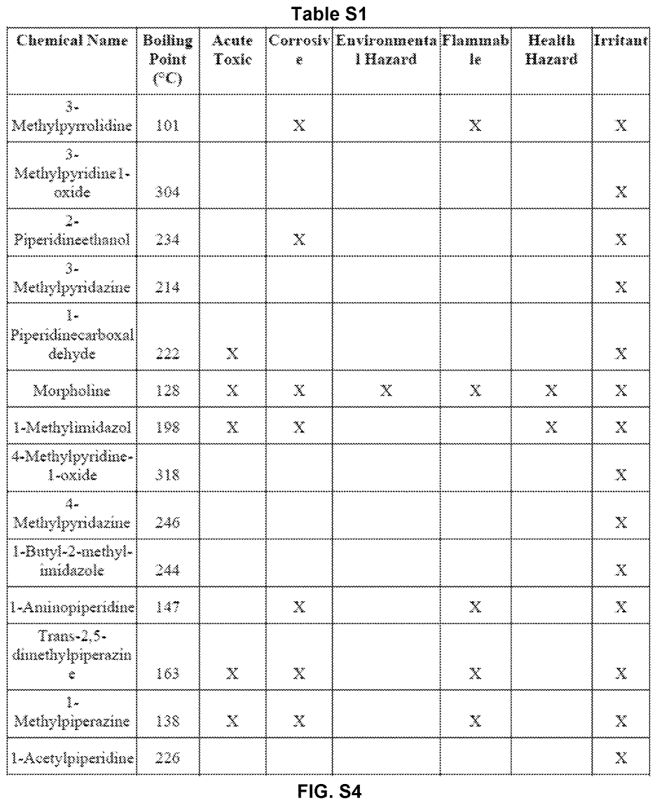

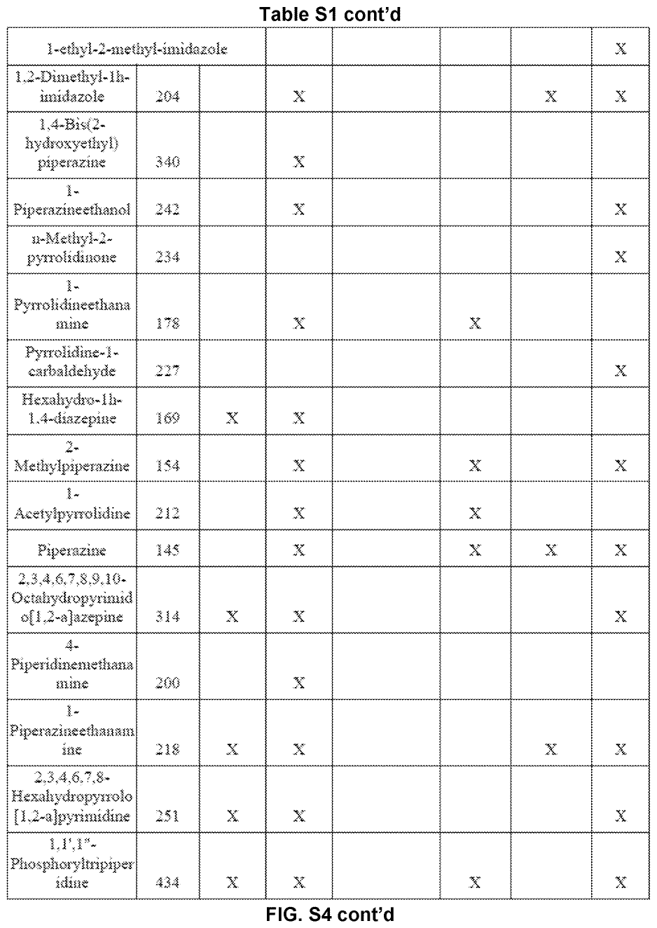

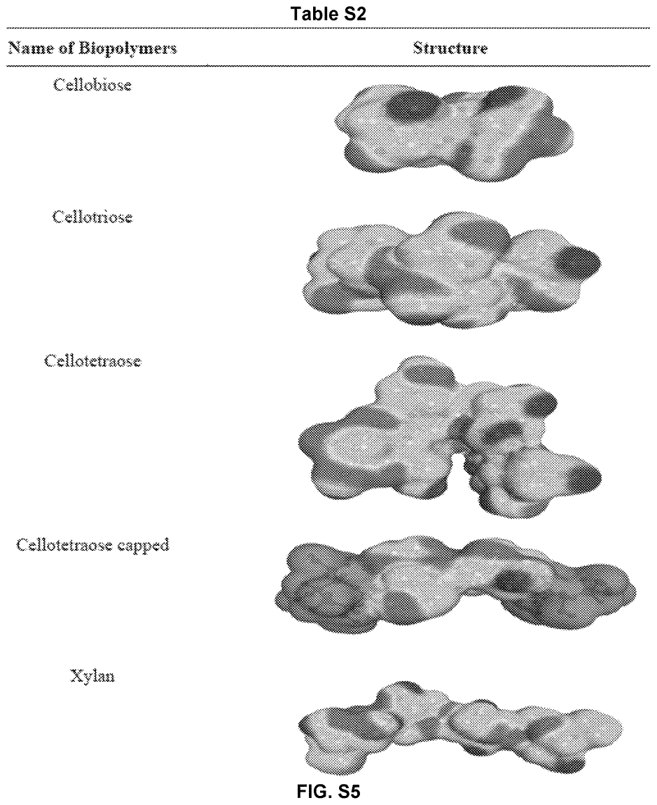

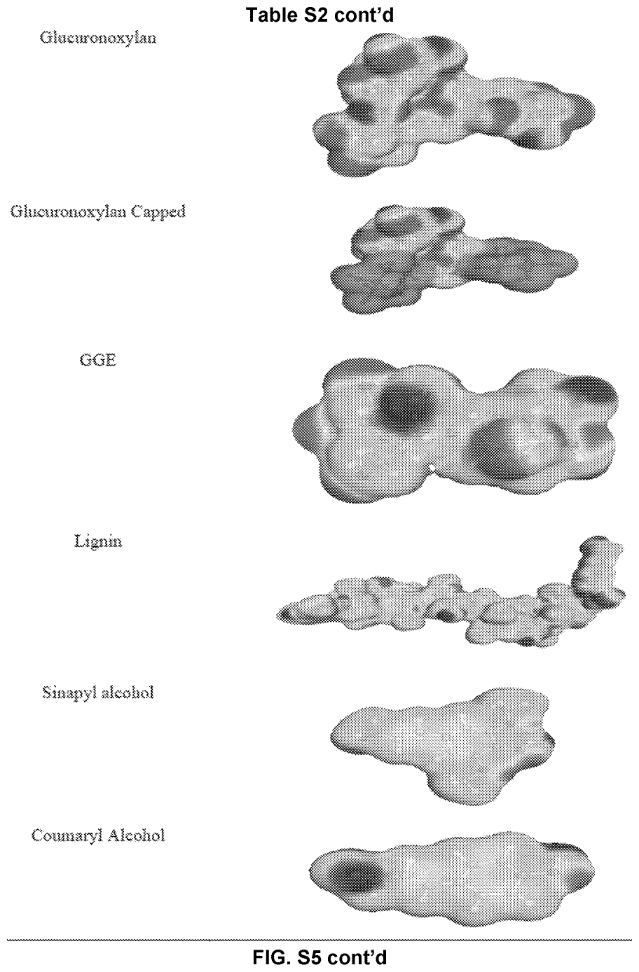

Notably, sorghum exhibited substantial lignin removal, reaching approximately 67% even at the mild temperature of 40° C., demonstrating the strong potential of piperazineethanamine-based pretreatment for efficient processing of herbaceous biomass. This highlights the promise of this approach as an energy-efficient and effective strategy for enhancing biomass deconstruction in grasses, while also indicating opportunities for further optimization when applied to more recalcitrant softwood systems like pine. See FIG. 13.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to deconstruct a biomass: the method comprising: (a) introducing a solvent comprising a cyclic amine, or a mixture of cyclic amines, to a biomass to dissolve at least part of solid biomass, or a part of a lignin, cellulose, and/or hemicellulose of the solid biomass, in the solvent.

2. The method of claim 1, further comprising: (b) introducing an enzyme and/or a microbe to the solubilized biomass mixture such that the enzyme and/or microbe produces a sugar from the solubilized biomass mixture.

3. The method of claim 2 further comprising: (c) separating the sugar from the solubilized biomass mixture.

4. The method of claim 3 further comprising: (d) separating the lignin from the solubilized biomass mixture.

5. The method of claim 1, wherein the cyclic amine cyclic amine comprises: (a) a 5- to 10-membered ring structure; (b) 1, 2, 3, or 4 nitrogen, oxygen, or sulfur atoms in a ring structure, wherein there is at least one nitrogen atom in the ring structure; (c) the ring structure has 0, 1, 2, 3, or 4 double-bonds; (d) none, or one or more carbon or nitrogen atoms in the ring structure is bonded to a R group, wherein each R is independently —H, —NH₂, alkyl, alkenyl, alkynyl, aryl, alkyl amine, alkenyl amine, alkynyl amine, or aryl amine, and optionally a ring or R comprises a carbonyl group, and optionally R is, or comprises, an amino group, carbonyl group, hydroxyl group, and/or carboxylic acid group; (e) optionally an oxygen atom bound to a nitrogen in the form of an oxide, and (f) optionally any carbon atom in the ring structure is bonded to a ═O.

6. The method of claim 5, wherein the cyclic amine has a chemical structure of: 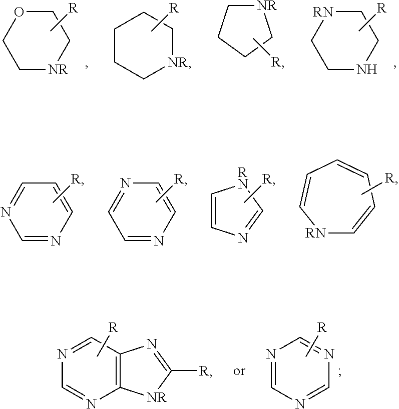
wherein each R is independent of any other R in the cyclic amine, and any 2 hydrogen atoms in a ring structure or in any R is replaced with a ═O. In some embodiments, each R is independently —H, —NH₂, alkyl, alkenyl, alkynyl, aryl, alkyl amine, alkenyl amine, alkynyl amine, or aryl amine, and optionally a ring or R comprises a carbonyl group, and optionally R is, or comprises, an amino group, carbonyl group, hydroxyl group, and/or carboxylic acid group, and when the cyclic amine is a purine, the R bonded to a nitrogen heteroatom within a ring can be bonded to any one of four nitrogen heteroatoms.

7. The method of claim 1, wherein the introducing step comprises dissolving at least part of cellulose of the solid biomass, and the cyclic amine comprises (1) an oxygen atom bound to a nitrogen in the form of an oxide, (2) a carbonyl group, (3) there is no oxygen atom present as a ring-heteroatom, and/or (4) a five or six membered ring comprising two or more nitrogen atoms as ring-heteroatoms; and/or the cyclic amine is soluble in water.

8. The method of claim 7, wherein the cyclic amine comprises (1) an oxygen atom bound to a nitrogen in the form of an oxide, or (2) a carbonyl group.

9. The method of claim 8, wherein the cyclic amine comprises an oxygen atom bound to a nitrogen in the form of an oxide.

10. The method of claim 9, wherein the cyclic amine is

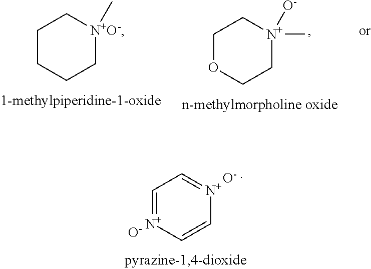

11. The method of claim 8, wherein the cyclic amine comprises a carbonyl group.

12. The method of claim 11, wherein the cyclic amine is a 5-member or 6-member ring.

13. The method of claim 12, wherein the cyclic amine is isonipecoticacid 2,5-piperazinedione

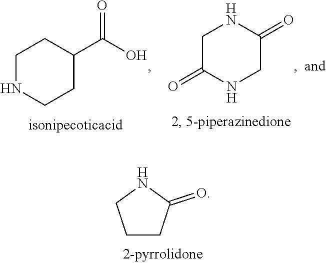 [SMILES: O=C(O)C1CCNCC1 ; O=C1CNC(=O)CN1 ; O=C1CCCN1]

14. The method of claim 7, wherein the cyclic amine has no oxygen atom present as a ring-heteroatom.

15. The method of claim 7, wherein the cyclic amine comprises a five or six membered ring comprising two or more nitrogen atoms as ring-heteroatoms.

16. The method of claim 15, wherein the cyclic amine is

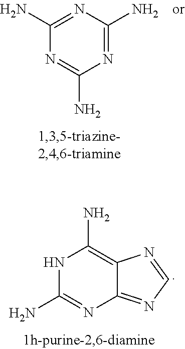 [SMILES: Nc1nc(N)nc(N)n1 ; Nc1nc2ncnc-2c(N)[nH]1]

17. The method of claim 7, wherein the cyclic amine is soluble in water.

18. The method of claim 1, wherein the introducing step comprises dissolving at least part of lignin of the solid biomass, and the cyclic amine has a pKa value within the range of about 8 to about 15.

19. The method of claim 18, wherein the cyclic amine has a pKa value within the range of about 9 to about 12.
